(19) United States
(12) Patent Application Publication
(10) Pub. No.: US 2026/0306371 A1
(43) Pub. Date: Oct. 8, 2026

(54) METHOD OF MANUFACTURING AN AEROSOL ANTIPERSPIRANT PRODUCT

(71) Applicant: THE PROCTER & GAMBLE COMPANY, Cincinnati, OH (US)

(72) Inventors: Ke Ming QUAN, West Chester, OH (US); Jianwei ZHANG, West Chester, OH (US); Beverly Ann HERRE, Liberty, OH (US)

(21) Appl. No.: 19/640,683

(22) Filed: Apr. 7, 2026

Related U.S. Application Data
(60) Provisional application No. 63/784,480, filed on Apr. 7, 2025.

(51) Int. Cl. A61K 8/26; A61K 8/04; A61K 8/49; A61K 8/891; A61Q 15/00

(52) U.S. Cl. CPC A61K 8/26; A61K 8/046; A61K 8/4973; A61K 8/891; A61Q 15/00; A61K 2800/805

(57) ABSTRACT

A method of making an antiperspirant composition by first combining a group of components that includes the antiperspirant active, a hydrophobically-modified clay, an anhydrous solvent, and a clay activator in a stream. Then, the stream is transferred through an apparatus for mixing liquids by producing shear and cavitation. The apparatus includes a mixing chamber and an element adjacent to the entrance of the mixing chamber. The element has an orifice that is configured to produce a share or cavitation and where the pressure at the orifice is greater than 500 psig. The apparatus increases the viscosity of the stream that forms that antiperspirant neuter. Finally, one or more late product differentiation materials to form the antiperspirant composition.

### FIELD OF THE INVENTION

The present disclosure is directed to the field of personal care products, specifically to the manufacturing process of aerosol antiperspirant products. More particularly, the present disclosure pertains to an improved method for enhancing the structural integrity and stability of aerosol antiperspirant compositions.

### BACKGROUND OF THE INVENTION

Aerosol antiperspirant products are effective and convenient. These products typically contain an antiperspirant composition that includes an active antiperspirant ingredient and other formulation components and a propellant. However, a common challenge with these products is that the antiperspirant composition loses viscosity and structure, which causes the antiperspirant active to fall out of solution and form a compacted cake at the bottom of the can.

Currently, aerosol antiperspirant compositions generally include clays, to impart desirable properties such as improved flow behavior, enhanced stability, and reduced clogging of dispensing mechanisms. However, the use of clays in the existing method of production often fails to provide the product with the required structural integrity, leading to sedimentation, which can lead to the formation of a compacted cake.

The inadequate structural stability of the aerosol antiperspirant composition can lead to several issues with consumer acceptance of aerosol antiperspirant products. Firstly, the presence of sedimentation within the product can cause an uneven distribution of active ingredients, resulting in inconsistent performance. Moreover, the sedimentation can lead to difficulties in product dispensing, diminishing the overall user experience.

Therefore, there is a need for an improved method for manufacturing aerosol antiperspirant products that enhances the structural integrity and stability of the antiperspirant composition, thereby minimizing sedimentation and compacted cake formation.

### SUMMARY OF THE INVENTION

A method of making an antiperspirant composition, the method comprising: (a) combining a first group of components comprising an antiperspirant active, a hydrophobically-modified clay, an anhydrous solvent, and a clay activator in a stream; (b) transferring the stream through an apparatus for mixing liquids by producing shear and cavitation, said apparatus comprising: (i) a mixing chamber, said mixing chamber comprising an entrance, at least one inlet, and an outlet; and (ii) an element with an orifice therein, said element being located adjacent the entrance of said mixing chamber, wherein said orifice is configured to produce shear or cavitation in the second stream, wherein the pressure at the orifice is greater than 500 psig; wherein the apparatus increases the viscosity of the stream forming the antiperspirant neuter; (c) adding one or more late product differentiation materials to form the antiperspirant composition.

### BRIEF DESCRIPTION OF THE DRAWINGS

While the specification concludes with claims particularly pointing out and distinctly claiming the subject matter of the present invention, it is believed that the invention can be more readily understood from the following description taken in connection with the accompanying drawings, in which:

FIG. 1 is a flowchart showing a method of manufacturing antiperspirant compositions and products with a single stream process;

FIG. 2 is a flowchart showing a method of manufacturing antiperspirant compositions and products with a dual stream process;

FIG. 3 is a partially fragmented cross-sectional view of one embodiment of an apparatus for mixing by producing shear and/or cavitation;

FIG. 4 is an enlarged perspective view of one embodiment of an element with an orifice therein for use in the apparatus shown in FIG. 3;

FIG. 5 is a cross-section of the element shown in FIG. 4 taken along line 3-3 of FIG. 4.

FIG. 6 is an enlarged perspective view of one embodiment of a blade for use in the apparatus shown in FIG. 3;

FIG. 7 is a simplified fragmented schematic cross-sectional view taken from an angle similar to that of FIG. 5 showing an orifice component comprising a composite of different materials;

FIG. 8A is a simplified cross-sectional view of a blade shown in a partially fragmented holder for the same in which the blade comprises a composite of different materials;

FIG. 8B is a simplified cross-sectional view similar to FIG. 8A showing another embodiment of a blade comprising a composite of different materials;

FIG. 9 is a three-dimensional graph showing the resonant frequency of a diamond blade as a function of blade thickness and length;

FIG. 10 is a three-dimensional graph showing the resonant frequency of a diamond blade as a function of blade thickness and aspect ratio (length/width);

FIG. 11 is a graph showing how the viscosity of the antiperspirant neuter changes under pressure; and

FIG. 12 are x-ray photographs of antiperspirant products made with the current process and the inventive process after 10 weeks of storage and after shaking various times.

### DETAILED DESCRIPTION OF THE INVENTION

Aerosol antiperspirant products are effective and convenient for reducing or eliminating body odor from various areas of the body, including underarms, privates, underboobs, feet, chest, legs, stomach, groin, neck, arms, and hands. While perspiration is odorless, it contains natural oils that can be a nutrient source for bacteria living on the skin. These bacteria interact with the natural oils, converting them into odor producing compounds. Antiperspirant compositions can contain an active, such as an aluminum salt, that reacts with the electrolytes in perspiration to form a plug in the ducts of sweat glands. The plugs prevent perspiration from exiting the duct, thereby depriving the bacteria of water and a food source. Antiperspirant compositions may be applied to the skin in an aerosol spray. Various examples of antiperspirant spray products and devices are described in U.S. Pat. Nos. 4,152,416; 4,806,338; 4,840,786; 4,904,463; 4,935,224; 5,298,236; 5,605,682; 5,814,309; and 7,815,899; and EP 674,899; WO 96/04884; and WO 2004/014330.

Aerosol antiperspirants products usually come in a container that holds both a propellant and an antiperspirant (AP) composition that contains an active ingredient along with other components. However, a common problem with these products is that the antiperspirant product can lose structure and viscosity, which can lead to undesirable sedimentation and/or a compacted cake inside the container.

Users of aerosol sprays are accustomed to shaking the container before use to mix the antiperspirant product and achieve a consistent, even spray. However, if there is a lot of sedimentation and/or a compacted cake inside the container, the user may have to shake excessively or vigorously. Even then, the spray device may become clogged or otherwise difficult to use, which is frustrating for consumers.

The proposed method aims to enhance the structural integrity and stability of the antiperspirant composition, thereby minimizing sedimentation and compacted cake formation.

Many antiperspirant compositions intended for use in an aerosol dispenser use suspending or bulking agents, such as clay-based materials, to help build the composition's structure. These clay-based materials are combined with other components, such as carriers, antiperspirant actives, skin feel modifiers, clay activators, masking agents, perfumes, and other materials, into a bulk antiperspirant composition, which is then milled to delaminate the clay and form the antiperspirant composition. This milling process (a machining process using rotary cutters) raises the viscosity of the antiperspirant composition. After milling, the antiperspirant composition is typically transferred to a storage tank where it undergoes continuous mixing to prevent product separation and to ensure batch homogeneity. The antiperspirant composition is then filled into individual spray devices and propellant is added to the composition in the spray device, thus forming an antiperspirant product. The consumer typically shakes the spray device before use, which is sufficient for the aerosol product to not choke or clog the spray device or otherwise distract from the consumer spray-on experience.

However, in some instances, the antiperspirant compositions may become damaged by the standard milling and filling processes, described above. The inventors have observed that some liquid carriers do not easily wet the clay, which causes the viscosity of the antiperspirant composition to quickly decrease under normal mixing conditions in the surge tank. Formulas that contain cyclopentasiloxane usually do not experience significant compaction issues. This is because cyclopentasiloxane helps prevent the powder particles from sticking together, making it easy to shake lose any settling or compaction that does occur before use. However, some consumers prefer antiperspirant products that do not contain cyclopentasiloxane and without this ingredient, the antiperspirant tends to be less structured and has a lower viscosity, which results in quicker sedimentation and the formation of a compacted layer. When this composition is put into a spray device and the propellant is added, it may require more vigorous shaking than what a typical consumer would do to break up the compacted layer and sediment. As a result, the spray device can become clogged, negatively affecting the consumer experience.

### Method of Manufacture

The inventors found that the semi-continuous manufacturing process, such as the one described herein, can help to more fully exfoliate the clay. This results in an antiperspirant product that maintains its structure and viscosity throughout its shelf life, ultimately reducing the likelihood of clogging in the spray device.

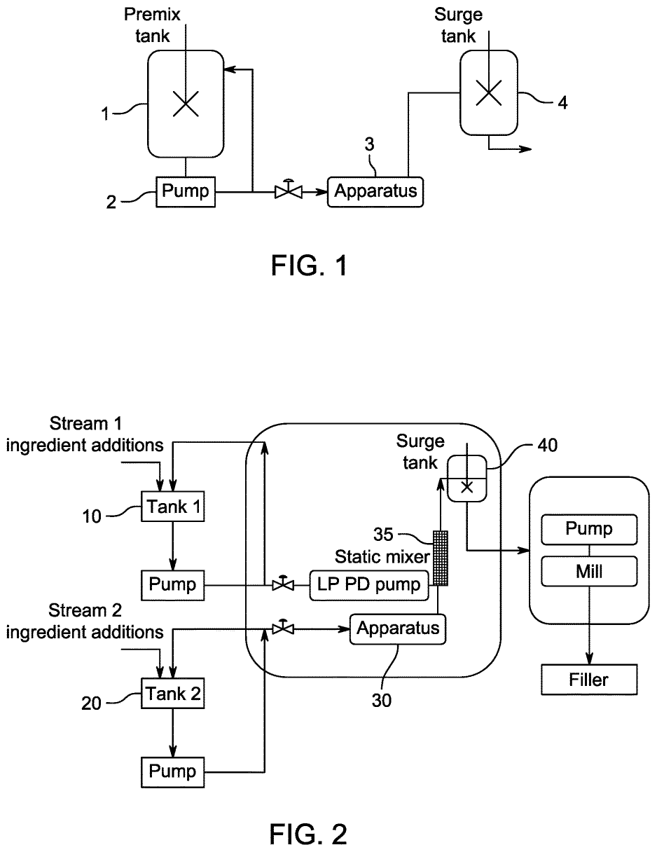

FIG. 1 shows a semi-continuous single stream process. First, an antiperspirant (AP) premix is made by adding all the components of the AP composition except for perfumes and other late product differentiation components. Components of the AP premix may include an AP active, hydrophobically-modified clay, anhydrous solvent, and a clay activator. The components are placed into premix tank 1 and continuously mixed using an appropriate mixing device, such as a pitched blade agitator, to obtain and maintain a homogenous AP premix, without applying too much shear.

When desired, such as when the AP neuter is needed on the packaging line, pump 2 transfers AP premix into apparatus 3 for milling, where the shear and cavitation will fully exfoliate the clay and produces a structured composition referred to as the AP neuter. Apparatus 3 can have an orifice that enables mechanical energy from the AP premix passing through the orifice to be effective for delamination and/or exfoliation to form the AP neuter. After passing through the apparatus 3, the AP neuter can flow into surge tank 4, where perfumes and other late product differentiation components can be added to create the final AP composition. The contents of surge tank 4 can be mixed, without shear, to maintain homogeneity and structure of the AP composition. Finally, the AP composition can be transferred from surge tank 4 to the packaging line, where it is placed into containers and combined with the aerosol propellant, resulting in the finished aerosol AP product.

Alternatively, a semi-continuous dual stream manufacturing process can be used to make the antiperspirant composition, as shown in FIG. 2. A first group of components may be combined in a first stream 1, which may include late product differentiation components, such as primary and secondary perfumes in powder form, such as starch-based encapsulates, along with an anhydrous solvent, are in tank 10. A second group of components may be combined in a second stream 2, which may include a hydrophobically-modified clay, a non-aqueous solvent, an anhydrous solvent, and/or a clay activator. The second stream 2 may be substantially free of water or it could be free of water. The second stream 2 components can be combined in tank 20, which is then sent into the apparatus 30 for milling. Apparatus 30 can have an orifice that can provide the mechanical energy to delaminate and/or exfoliate the AP premix to form the structured AP neuter. The one or more antiperspirant actives can be in first stream 1 and/or second stream 2. The anhydrous solvent can be the same in the first and second stream or it can be different. Apparatus 30 uses shear and/or cavitation to exfoliate the clay. Upon exiting apparatus 30, stream 2 may be combined with stream 1 in a vessel with a static mixer 35, creating an antiperspirant composition. Finally, the antiperspirant composition can be transferred from surge tank 40 and then transferred to the filler and packaging line, where the antiperspirant composition is placed into containers and combined with the aerosol propellant, resulting in the finished aerosol AP product.

Other ways to manufacture antiperspirant products may include adding a liquid activation enhancer into the formulation, such as discussed, for example, in U.S. Pat. No. 10,787,283. Certain liquid materials may help maintain and/or promote the clay bulking and suspending benefit in an antiperspirant composition that comprises a non-volatile silicone liquid, and optionally a liquid fragrance material, by facilitating increased interaction or loose bonding between the non-volatile silicone fluid and the clay material. It is believed that the increased interaction may be facilitated, in some instances, when the liquid activation enhancer is soluble in the non-volatile silicone and has a Hansen Solubility Parameter for Hydrogen Bonding, δₕ, between about 2 MPa^{1/2} and about 6 MPa^{1/2}. However, in some embodiments, the apparatus of the present invention renders the liquid activation enhancer unneeded, as the apparatus can provide sufficient exfoliation of the clay.

For the apparatus, the pressure at the orifice may be greater than about 500 psig, alternatively from about 500 psig to about 5000 psig, alternatively from about 1000 psig to about 4500 psig, alternatively from about 2000 psig to about 4000 psig, and alternatively from about 3000 psig to about 4000 psig. The corresponding energy density for the apparatus may be from about 3.45×10⁶ J/m³ to about 3.45×10⁷ J/m³. For formulations for which the density is close to 1 g/ml, the energy density may be expressed as from about 3.45 J/g to about 34.5 J/g. For the apparatus, the size of the orifice may be from about 0.0007 in² to about 0.1 in²; from about 0.0007 in² to about 0.02 in², from about 0.007 in² to about 0.01 in², and in some embodiments from about 0.007 in² to about 0.1 in².

### Apparatus

The apparatus is similar to that discussed in U.S. Pat. Nos. 8,517,595 and 8,322,910. The ability of the apparatus and method to induce shear may not only be useful for mixing but may also be useful for dispersion of solid particles in liquids and in breaking up solid particles. The ability of the apparatus and method to induce shear and/or produce cavitation may also be useful for droplet and/or vesicle formation.

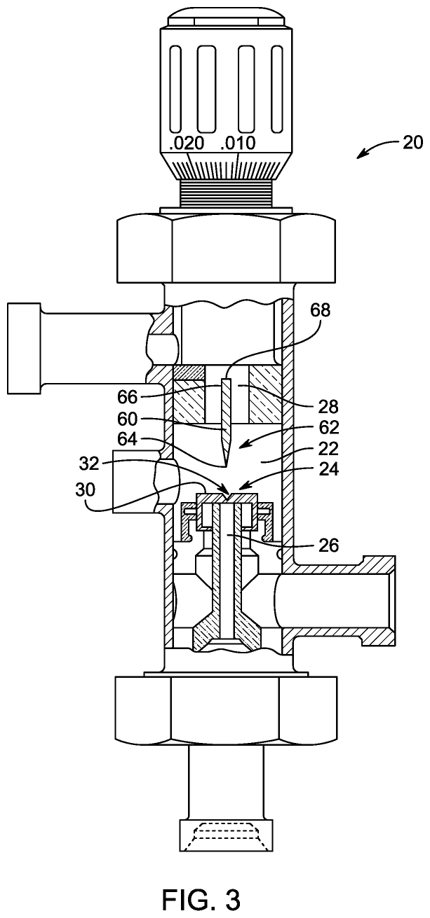

FIG. 3 shows one non-limiting embodiment of an apparatus 20 for mixing by producing shear and/or cavitation. As shown in FIG. 3, the apparatus 20 comprises: a mixing and/or cavitation chamber 22 which comprises an entrance 24, at least one inlet 26 and an outlet 28; and an element 30 or structure such as an orifice component with an orifice 32 therein. The element 30 is located adjacent the entrance 24 of the mixing and/or cavitation chamber 22. The apparatus 20 may further comprise a blade 60, such as a knife-like blade, disposed in the mixing and/or cavitation chamber 22 opposite the element 30 with an orifice 32 therein.

The apparatus 20 can comprise a SONOLATOR® high pressure homogenizer available from Sonic Corp. of Stratford, CT, U.S.A. as shown; or, a Gaulin homogenizer available from Invensys APV, Lake Mills, WI, U.S.A.; a Microfluidizer® fluid material processor available from Microfluidics Corp. of Newton, MA, U.S.A.; an atomizer; or, any other suitable apparatus. SONOLATOR® high pressure homogenizers are described in U.S. Pat. No. 3,176,964 issued to Cottell, et al. and U.S. Pat. No. 3,926,413 issued to D'Urso. Although the following discussion is expressed primarily in terms of the components for the SONOLATOR® high pressure homogenizer, it should be understood that the materials described herein can be used for the parts that are subject to wear in any type of high pressure mixing or homogenizing device, including but not limited to the valve and/or disk used in other high-pressure homogenizers. It may be desirable for the apparatus described herein, and the components thereof, to exclude those used in water jet cutting devices and sand blasting devices. Thus, it is desirable for the apparatus to be capable of mixing the materials that are fed into the apparatus, rather than merely spraying water. This mixing can take place inside a mixing chamber, such as the cavitation chamber 22.

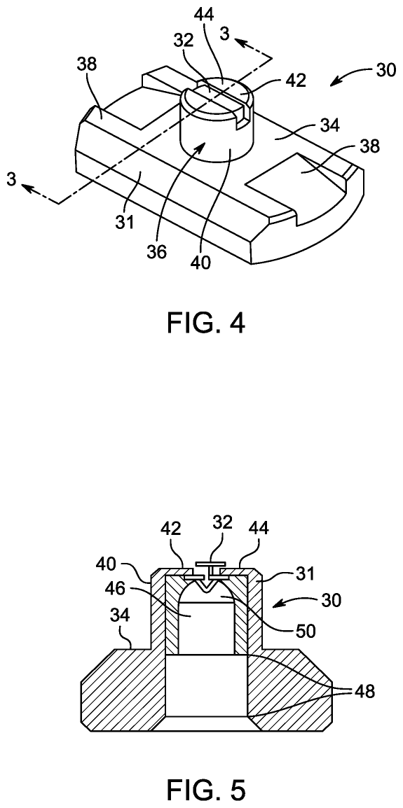

The element 30 with the orifice 32 therein can be in any suitable configuration. In FIG. 4, the element 30 comprises a housing 31 comprising a plate-like portion 34 with a protuberance 36 in the central region of the plate-like portion 34. The plate-like portion 34 defines sides or wings 38 to the side of the protuberance 36. FIG. 4 shows that the protuberance 36 comprises side walls 40 having a top or front surface 42 joined thereto. The front surface 42 of the protuberance 36 has the orifice 32 formed therein and portions 44 surrounding the orifice 32.

The term “joined”, as used herein, encompasses configurations in which an element is directly secured to another element by affixing the element directly to the other element; configurations in which the element is indirectly secured to the other element by affixing the element to intermediate member(s) which in turn are affixed to the other element; and configurations in which one element is integral with another element, i.e., one element is essentially part of the other element.

The orifice 32 can be in any suitable configuration. Suitable configurations can include, but are not limited to: slot-shaped, eye-shaped, elliptically-shaped, triangular, square, rectangular, in the shape of any other polygon, or circular. In some embodiments, as shown in FIG. 2, it may be desirable for the width of the orifice to exceed the height of the orifice. The width of the orifice 32 may be any multiple of the height of the orifice including, but not limited to: 1.1, 1.2, 1.3, 1.4, 1.5, 2, . . . , 2.5, 3, 3.5, . . . , etc. up to 100 or more times the height of the orifice. The orifice 32 can be of any suitable width including, but not limited to, up to about 1 inch (2.54 cm), or more. The orifice 32 can have any suitable height including, but not limited to, up to about 0.5 inch (about 1.3 cm), or more.

FIG. 3 shows that the element 30 with the orifice 32 therein can comprise one or more components or an orifice component system. For example, in the embodiment shown in FIG. 5, the element 30 with the orifice 32 therein comprises a housing 31 with a hollow protuberance 36, and the hollow protuberance 36 has an insert 46 therein. The insert 46 can be in any suitable configuration. In the embodiment shown in FIG. 5, the insert 46 is a bullet-shaped element that has one or more flange portions 48 at the mid-portion and/or rear part of the insert 46. The insert 46 has an interior passageway for transport of liquid therethrough and a nozzle portion 50 at the front of the insert. This interior passageway is shown in greater detail in FIG. 7. FIG. 7 also shows that the portions 44 surrounding the orifice 32 form a wall 52 that becomes thinner in at least some places as the orifice 32 is approached. Numerous other configurations for the insert 46 are possible including, but not limited to flat, plate-like configurations. The insert 46 may be made of a harder material than other portions or components of the structure comprising the element 30. The insert 46 is used so that the other larger portions or components of the element 30 can be made from a less hard, and less expensive material.

The element 30, and the components thereof, can be made of any suitable material or materials. Suitable materials can include titanium, stainless steel, tool steel, cemented tungsten carbide, ceramics (including oxide ceramic materials and non-oxide ceramic materials), silicon nitride (Si₃N₄), titanium nitride (TiN), aluminum oxide (Al₂O₃) (which in single crystal form is known as sapphire), silicon carbide (SiC), titanium carbide (TiC), boron carbide (B₄C), titanium diboride (TiB₂), boron oxide (B₆O), cubic boron nitride (cBN) (including polycrystalline cBN and epitaxially-grown cBN), cubic BC₂N, diamond-like carbon (DLC), diamond (e.g., bulk diamond) (natural and synthetic), composites of diamond and/or cubic boron nitride, and coatings of any of the above materials, including but not limited to diamond-coated materials (polycrystalline diamond-coated materials and epitaxially-grown diamond coatings) and diamond-like carbon. Other suitable materials include combinations of incompressible transition metals, which are relatively soft (such as osmium, rhenium, and ruthenium), with small covalent main group elements having short bonds (such as boron, carbon, nitrogen, or oxygen) to make the metals hard, producing materials such as osmium diboride (OsB₂), rhenium diboride (ReB₂) and ruthenium diboride (RuB₂). Other materials potentially of interest include: osmium carbide (OsC), osmium nitride (OsN), osmium dioxide (OsO₂), rhenium carbide (ReC), rhenium nitride (ReN), rhenium dioxide (ReO₂), ruthenium carbide (RuC), ruthenium nitride (RuN), and ruthenium dioxide (RuO₂). Some of these materials may be formed in the absence of high pressure, making them more economical to produce. These latter materials are described in the following articles: R. W. Cumberland, et al., “Osmium diboride, an ultra-incompressible, hard material”, J. Am. Chem. Soc., 127, 7264 (2005); R. B. Kaner, et al., “Designing superhard materials”, Science, 308, 1268 (2005); H. Chung, et al., “Synthesis of Ultra-Incompressible Superhard Rhenium Diboride at Ambient Pressure”, Science, Vol. 316, Apr. 20, 2007; and, “Materials, Taking a Super-hard Approach”, R&D Magazine, May 2007, published by Advantage Business Media, Rockaway, NJ, U.S.A. The Vickers hardness Hv of several of these materials are provided in Table 1 below. According to J. Mater, Res., Vol. 17, No. 12, December 2002, Materials Research Society, materials with a Vickers hardness greater than or equal to 40 GPa are considered superhard materials.

TABLE 1
Vickers Hardness of Various Materials

| | Approximate Vickers hardness |
|---|---|
| Material | Giga Pascals (Gpa) |
| Tool steel | 4.5-6.5 |
| Cemented Carbide Tooling¹ | 11-19 |
| Silicon Nitride (Si₃N₄) | 22 |
| Titanium Nitride (TiN) | 22-31 |
| Aluminum Oxide (Al₂O₃) (Sapphire) | 23 |
| Silicon Carbide (SiC) | 28 |
| Boron Carbide (B₄C) | 30 |
| Titanium Diboride (TiB₂) | 32 |
| Boron Oxide (B₆O) | 35 |
| Rhenium Diboride (ReB₂) | 30-48 |
| Cubic Boron Nitride (cBN) | 62 |
| Cubic BC₂N | 75 |
| Diamond | 95 |

¹Microhardness Material Hardness Measurement, www.brycoat.com/hardness.htm

In FIGS. 4 and 5, the element 30 comprises a housing 31 and an insert 46. It may be desirable for at least the nozzle portion 50 at the front of the insert 46 to be made of a material having a Vickers hardness of greater than or equal to about 20 GPa because this is the portion of the orifice component system that is subject to the greatest forces when liquids and/or other material is sprayed through the nozzle portion 50. Alternatively, it may be desirable for the entire insert 46 to be made of a material having a hardness in the hardness ranges specified above. Alternatively, where there is a single component orifice component system (that is, with no separate insert), it may be desirable for at least the portions 44 surrounding the orifice 32 (which would be subjected to the greatest forces) to be made of a material having a hardness in the hardness ranges specified above.

Thus, in certain non-limiting embodiments, the portions 44 of the element 30 surrounding the orifice 32 may be provided with a Vickers hardness of greater than or equal to about 20 GPa.

The portions 44 surrounding the orifice 32 may be provided with a Vickers hardness in any increment of 1 Vickers hardness unit above 20 GPa. All or a portion of any of the components of the orifice component system can be made out of one of the materials described herein, and at least some of such components can be partially or entirely coated with a material described herein which has a higher hardness.

Materials having a Vickers hardness of greater than or equal to about 20 GPa which are useful herein include, but are not limited to: silicon nitride, titanium nitride, aluminum oxide, silicon carbide, titanium carbide, boron carbide, titanium diboride, boron oxide, rhenium diboride, cubic boron nitride, cubic BC₂N, diamond-like carbon, diamond (e.g., bulk diamond) (natural and synthetic), and composites of diamond and/or cubic boron nitride, and coatings of any of the above materials, including but not limited to diamond-coated materials and diamond-like carbon. Materials have also been theorized (e.g., carbon nitrite) which are expected by some people to have a hardness similar to that of diamond. Such materials, if and when they become available, may also be suitable for use herein. Several of these materials are described in greater detail below.

Cubic boron nitride (cBN) is a synthetic material, which is available both in bulk form (as a polycrystalline structure and as a single crystal), and in the form of coatings (polycrystalline coatings and epitaxially-grown coatings).

Diamond like carbon (DLC) is characterized by both SP2 and SP3 bond structures, and as a result, tends to be amorphous. Diamond like carbon is typically only available in the form of coatings (rather than bulk form), and is highly wear resistant, and very stable chemically against erosion and corrosion.

Natural and synthetic diamonds are characterized by SP3 bond structures. Synthetic (man-made) diamond include polycrystalline diamond (PCD) and single crystal diamond, and coatings. Polycrystalline diamond (PCD) can include bulk diamond, which comprises a three-dimensional structure of many crystals that are sintered together, and polycrystalline diamond coatings that are deposited on an appropriate substrate.

Composites of diamond and cBN typically comprise composites of polycrystalline diamond (PCD) and a polycrystalline cBN. The polycrystalline diamond (PCD) used in such composites can be natural or synthetic. Suitable composites can comprise any suitable proportions of PCD and cBN, from 1-99% PCD to 1-99% cBN. Composites of diamond and cBN are typically formed at high temperature and high pressure by sintering particles of polycrystalline diamond (PCD) and cBN together with binders such as nickel, cobalt, or iron.

Element 30, and the components thereof, can be formed in any suitable manner. Any of the components of the orifice component system 30 can be formed from solid pieces of the materials described above which are available in bulk form. The components may also be formed of a solid piece of one of the materials specified above, which is coated over at least a portion of its surface with one or more different materials specified above. In these embodiments, or in other embodiments, the orifice component system 30 can be formed from more than one piece. In this latter case, the different pieces can be formed from different materials. For example, in one embodiment, the orifice 32 can be provided in an insert, such as insert 46, that is cut out of a diamond using a laser or hot wire diamond cutter, diamond-based cutting tools, and optionally polished using diamond dust. This insert 46 can be inserted into or joined to a metal piece that forms the rest of the orifice component system, such as the housing 31. In other cases, the entire element 30, including the housing 31 can be made of bulk diamond, and there need not be an insert.

FIG. 7 shows a portion of at least one of the components of the element 30 is made out of one of the materials described herein, and this portion can be partially or entirely coated with a material described herein which has a higher hardness. For instance, the inner portion, A, comprising the insert 46 of the element 30 can be made of: steel, titanium, or cemented tungsten carbide, and the outer portion B thereof can comprise a coating of cubic boron nitride (cBN) (in the form of either polycrystalline coatings and epitaxially-grown coatings), or a diamond coating. These and other suitable combinations of materials for such a composite structure are listed in, hereafter.

TABLE 2
Combinations of Materials in Composite
Orifice and Blade Structures

| Component A | Component B |
|---|---|
| Titanium | Silicon nitride, titanium nitride, aluminum |
| | oxide, silicon carbide, titanium carbide, |
| | boron carbide, titanium diboride, boron |
| | oxide, cubic boron nitride (cBN), cubic |
| | BC₂N, diamond-like carbon (DLC), |
| | diamond, and coatings of any of the above |
| | materials, including but not limited to |
| | diamond-coated materials (polycrystalline |
| | diamond-coated materials and epitaxially- |
| | grown diamond coatings), diamond-like |
| | carbon, and composites of diamond and |
| | cubic boron nitride |
| Stainless steel | Same as above |
| Tool steel | Same as above |
| Cemented Carbide Tooling | Same as above |
| Silicon Nitride (Si₃N₄) | Same as above, except silicon nitride |
| Titanium Nitride (TiN) | Same as above, except silicon nitride and |
| | titanium nitride |
| Aluminum Oxide (Al₂O₃) | Same as above, except silicon nitride, |
| (Sapphire) | titanium nitride, and aluminum oxide |
| Silicon Carbide (SiC) | Same as above, except silicon nitride, |
| | titanium nitride, aluminum oxide, and |
| | silicon carbide |
| Boron Carbide (B₄C) | Same as above, except silicon nitride, |
| | titanium nitride, aluminum oxide, silicon |
| | carbide, and boron carbide |
| Titanium Diboride (TiB₂) | Boron Oxide, Rhenium Diboride, Cubic |
| | Boron Nitride, Cubic BC₂N, and diamond |
| Boron Oxide (B₆O) | Rhenium Diboride, Cubic Boron Nitride, |
| | Cubic BC₂N, and diamond |
| Rhenium Diboride (ReB₂) | Cubic Boron Nitride, Cubic BC₂N, and |
| | diamond |
| Composites of Cubic | Cubic Boron Nitride, Cubic BC₂N, and |
| Boron Nitride (cBN), | diamond |
| Cubic BC₂N, and Diamond | |
| Cubic Boron Nitride (cBN) | Cubic BC₂N and diamond |
| Cubic BC₂N | Diamond |
| Diamond (single crystal | Diamond coating |
| and polycrystalline) | |

In other embodiments, the element 30 with the orifice 32 therein need not have a hardness at the level specified above. For example, in other embodiments, the element 30 with the orifice 32 therein can have portions surrounding the orifice 32 with a Vickers hardness of less than about 20 GPa.

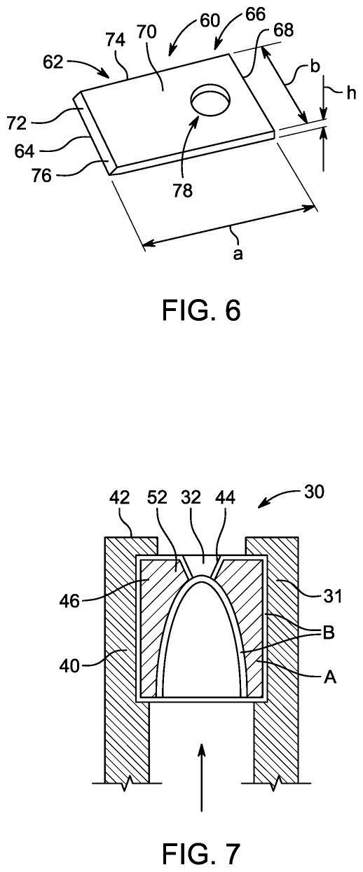

It can be desirable for the apparatus for mixing by producing shear and/or cavitation to comprise a blade 60. A blade 60 may be used, for example, if it is desired to use the apparatus 20 to form emulsions with a lower mean droplet size than if the blade was not present. As shown in FIG. 3, the blade 60 has a front portion 62 comprising a leading edge 64, and a rear portion 66 comprising a trailing edge 68. As shown in FIG. 6, the blade 60 also has an upper surface 70, a lower surface 72, and a thickness, h, measured between the upper and lower surfaces. In addition, the blade 60 has a pair of side edges 74 and a width, b, measured between the side edges 74.

The blade 60 can have any suitable configuration. As shown in FIG. 6, the blade can comprise a tapered portion 76 in which the thickness of the blade increases from the leading edge 64 in a direction from the leading edge 64 toward the trailing edge 68 along a portion of the distance between the leading edge 64 and the trailing edge 68. The blade 60 shown in FIG. 6 has a single tapered or sharpened edge forming its leading edge 64. It should be understood that in other embodiments, the blade 60 may have two, three, or four or more tapered or sharpened edges. For example, the blade 60 can have a rectangular, or square configuration, and any or all of its sides can be sharpened. Such a configuration may be useful so that the blade 60 can be inserted into the apparatus 20 with any of the sharpened edges oriented to form the leading edge 64 of the blade 60. This will multiply the useful life of the blade before it is necessary to repair the same.

The blade 60 can have any suitable dimensions. In certain embodiments, the blade 60 can range in size from as small as 1 mm long and 7 microns thick to as big as 50 cm long and over 100 mm thick. One non-limiting example of a small blade is about 5 mm long and 0.2 mm thick. A non-limiting example of a larger blade is 100 mm long and 100 mm thick.

As shown in FIG. 3, when the blade 60 is inserted into the apparatus 20, a portion of the rear portion of the blade 60 is clamped, or otherwise joined inside the apparatus so that its position is fixed. The blade 60 can be configured in any suitable manner so that it can be joined to the inside of the apparatus. As shown in FIG. 6, the rear portion 66 of the blade 60 can have at least one hole 78 therein for receiving a structure that passes through the hole. This hole and structure serves as at least part of the mechanism used to retain the blade 60 in place inside the apparatus 20. The blade 60 can also be joined to a holder which may be comprised of metal or another suitable material. The remainder of the blade 60, including the front portion 62 of the blade 60, is free and is cantilevered relative to the fixed portion.

The blade 60 can comprise any suitable material or materials. The blade desirably will comprise a material, or materials, that are chemically compatible with the fluids to be processed. (The same may also be desirable for the components of the orifice component system.) It may be desirable for the blade to be comprised at least partially of a material that is chemically resistant to one or more of the following conditions: low pH's (pH's below about 5); high pH's (pH's above about 9); salts (chloride ions); and oxidation.

Suitable materials for the blade 60 can include but are not limited to any material or materials described herein as being suitable for use in the orifice component system, and the components thereof. It should be understood, however, that the materials specified herein do not necessarily have all of the desired chemical resistance properties. The entire blade 60 may be comprised of one of the above materials.

Alternatively, a portion of the blade 60 may comprise one of the materials described herein as being suitable for use in the orifice component system, and another portion (or portions) of the blade 60 may comprise a different one of these materials. For example, in some cases, it may be desirable for a portion of the blade, such as the tapered portion 76, to comprise a harder material than the remainder of the blade 60. This may be desirable since the tapered portion 76 forms the leading edge 74 of the blade 60 and will be the portion of the blade subject to greatest wear during use. The remainder of the blade 60 can be comprised of some other material, such as a material that has one or more of the following properties: is less hard, less expensive, more ductile, or less brittle than the tapered portion 76.

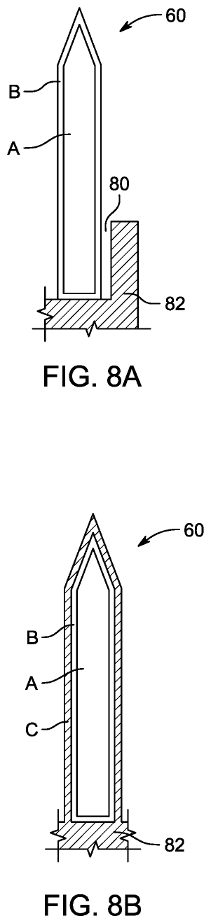

shows non-limiting examples of many of the different possible combinations of materials that can be used to construct one or more components of an element 30, or a composite blade 60. The letter “A” in Table 2 represents a portion of one or more components of the element 30, or a composite blade 60 structure shown in FIGS. 7 and 8A, respectively, that may be referred to as an “inner” portion or structure, and the letter “B” represents a component of the composite structure that may be referred to as an “outer” portion or coating. In one non-limiting example, the inner portion A of the blade 60 could comprise sintered PCD which has a diamond coating, B, formed by chemical vapor deposition.

As shown in FIG. 8A, it may further be desirable for the apparatus 20 to be provided with dampening material 80 between the blade 60 and the structure 82 to which the blade 60 is clamped. Material suitable for dampening includes, but is not limited to rubber, TEFLON® material, and indium foil.

As shown in FIG. 8B, it is also possible to provide more than one coating on all or a portion of the surfaces of the components of the orifice component system 30 or the blade 60. The blade 60 shown in FIG. 8B has a coating B thereon, and additionally has a second coating, C, disposed on the coating B. In this case, the coating B forms an intermediate coating. There can be any suitable number of coatings on the components of the composite orifice component system 30, or the composite blade 60. The coatings B and C can both comprise the same material; or, they may comprise any different materials described herein as being suitable for application in the form of coatings. Such additional coatings can be provided for any suitable purpose. For instance, if the coating C has the hardness desired, but it is not chemically or thermally compatible with the material forming the inner portion A, it may be desirable to form an intermediate coating B that is compatible with the material forming the inner portion A and the material of coating C so that coating C can be properly joined to the inner portion A.

In addition, any of the structures described herein can be repaired with any of the coatings described herein. For example, if a component of the orifice component system 30 or the blade 60 degrades through wear, it is possible to repair the worn component or blade with the coatings described herein (such as repairing the same with a diamond coating formed by chemical vapor deposition).

The blade 60, or various portions thereof, may have any suitable hardness. At least the tapered portion 76 of the blade 60 can be formed from a material with a hardness in the hardness ranges specified above. Alternatively, at least the tapered portion 76 of the blade 60 may be formed from a material with a Vickers hardness in any increment of 1 Vickers hardness unit above 20 GPa.

The remainder of the blade can comprise a material that has a Vickers hardness of less than 20 GPa. For instance, the tapered portion 76 of the blade 60 can be provided with a diamond coating, and the remainder of the blade 60 could be made of stainless steel.

Several non-limiting examples of methods of forming a blade 60 are described below. These include: forming a coated composite structure; forming a blade by coating layers of a material to form or build the final blade structure; and, forming a blade of a bulk material. The same techniques can be used to form components of the orifice component system, or components of other types of devices.

There are a number of methods of forming coated composite structures such as diamond coated structures, cBN coated structures, diamond composite coated structures, or structures coated with any of the other materials described herein which are capable of being applied in the form of coatings. Suitable methods include, but are not limited to: physical vapor deposition, chemical vapor deposition, and plasma deposition. In one non-limiting embodiment, a composite blade is formed from a stainless steel, titanium, or cemented tungsten carbide substrate that forms the interior of the blade, which is coated with a diamond coating, cBN, or diamond like carbon coating.

In another embodiment, a polycrystalline solid diamond blade 60 is made by coating a generally flat silicon single crystal substrate or wafer with diamond using plasma by conventional plasma deposition techniques, such as plasma chemical vapor deposition (CVD), or by hot filament deposition. In this method, the silicon substrate is initially seeded with small (approximately 1 micron in size) diamond nuclei by rubbing diamond paste or diamond powder onto the substrate. The diamond-coating approach can use saturated or non-saturated hydrocarbons, such as methane, acetylene, or ethylene in a hydrogen or in a noble gas (such as argon) environment and/or plasma environment to deposit the diamond-containing coating. The diamond-containing coating can be deposited layer-by-layer on the silicon substrate. After the thickness of diamond film reaches about 2 mm thick, the process is discontinued, and the silicon substrate can be removed. This process may be referred to as plasma-enhanced chemical vapor deposition (or “PECVD”). The silicon substrate can be removed by (wet or dry) etching, or by mechanical grinding. This will create a blade structure which is rough on both sides. The blade can be provided with a tapered leading edge by laser ablation and/or mechanical grinding. Chemical and/or mechanical means can then be used to polish the blade structure.

In other embodiments, the blade 60 can comprise a bulk material, such as bulk diamond material. Such a material can be formed in any suitable manner such as by high pressure and high temperature sintering in the presence of bonding elements such as cobalt, nickel, or iron using presses that form synthetic diamond from diamond dust. Forming the blade out of some materials, such as bulk diamond may be useful to provide the blade with more efficient acoustic vibration characteristics.

The acoustic properties may vary significantly from one material to another. Accordingly, the shape and size of the blade may need to differ depending on the material of the blade for a given resonant mode. Cavitation threshold (the minimum acoustic pressure at the onset of cavitation) is acoustic frequency dependent. At high ultrasonic frequencies, cavitation becomes increasingly more difficult. The maximum cavitation intensity occurs at low ultrasonic frequencies. It is desired to have the blade designed so that its resonant frequency is in the acoustic and low ultrasonic frequency range of from about 15 kHz to 100 kHz, or any narrower range of frequencies falling within such range including, but not limited to from about 18 kHz to 40 KHz.

The blade 60 is schematically illustrated as a rectangular plate in FIG. 6. Assuming (for simplicity) that the hole is not present, and one end 68 of the blade is clamped, and the other three sides are free. The plate has a length of a (from the clamping point), width of b and thickness of h. The natural frequency of the blade can be expressed as

$${f}_{i j} = {\frac{{k}_{i j}^{2}}{2 π {a}^{2}} [ \frac{E {h}^{2}}{12 ρ ( I - {v}^{2} )} ]}^{1 / 2} \qquad Eq . 1$$

where fᵢⱼ is the resonant frequency at mode indices (i,j), i being the mode number in length, j being the mode number in width; kᵢⱼ is a dimensionless frequency parameter; E is the modulus of elasticity, r is the density, and u is Poisson's ratio.
As a compression wave, the wave velocity C can be expressed as

$$C = \sqrt{\frac{E}{ρ}} \qquad Eq . 2$$

Substituting Eq.2 into Eq.1, the equation for resonant frequency becomes,

$${f}_{i j} = \frac{{k}_{i j}^{2}}{2 π {a}^{2}} {Ch [ \frac{1}{12 ( 1 - {v}^{2} )} ]}^{1 / 2} \qquad Eq . 3$$

The mechanical properties of several superhard materials, and cemented tungsten carbide are set forth in, below, for comparison. Cubic boron nitride and cubic BC₂N (c-BC₂N) are superhard materials having a hardness close to that of diamond. These materials are similar to diamond in that they are all: isotropic (their properties are the same in all directions), covalently bonded, and, all their bonds are SP3 bonds. There are differences in the mechanical properties of these materials depending on whether the material is in polycrystalline or in single crystal structures. However, the magnitude of the difference due to the state of each material is relatively small compared to the differences in these properties between the materials. The mechanical properties listed in below are the values in polycrystalline state, but the values are expected to approximate that of the single crystal state.

TABLE 3
Mechanical properties of the superhard materials: diamond,
c-BC₂N and cBN in comparison to cemented tungsten carbide.

| | Elastic | | Predicted |
|---|---|---|---|
| | modulus | Density | Acoustic velocity |
| Material | (GPa) | (gram/cm³) | (m/s) |
| Cemented | 630 | 14.900 | 6,502 |
| Tungsten Carbide | | | |
| (94% WC + 6% Co) | | | |
| Cemented | 470 | 12.900 | 6,036 |
| Tungsten Carbide | | | |
| (75% WC + 25% Co) | | | |
| cBN | 909 | 3.500 | 16,115 |
| c-BC₂N | 980 | 3.358 | 17,083 |
| Diamond | 1141 | 3.512 | 18,024 |

Due to its high elastic modulus (>900 MPa) and relatively low bulk density (3.512 g/cm³), diamond has the fastest acoustic velocity at 18,024 m/s. This compares to titanium 6,070 m/s, stainless steel of 5,900 m/s, water of 1,500 m/s and air of 310 m/s.

It may be desirable for the blade 60 to have an elastic modulus of greater than or equal to about 700, 750, 800, 850, or 900 GPa. It may be desirable for the blade to have a density of less than or equal to about any of the following: 12, 10, 9, 8, 7, 6, 5, 4, or 3.6 g/cm³. It may be desirable for the blade 60 to have or be made from a material having a predicted acoustic velocity greater than or equal to about any of the following: 7,640, 8,000, 9,000, 10,000, 11,000, 12,000, 13,000, 14,000, 15,000, or 16,000 m/s.

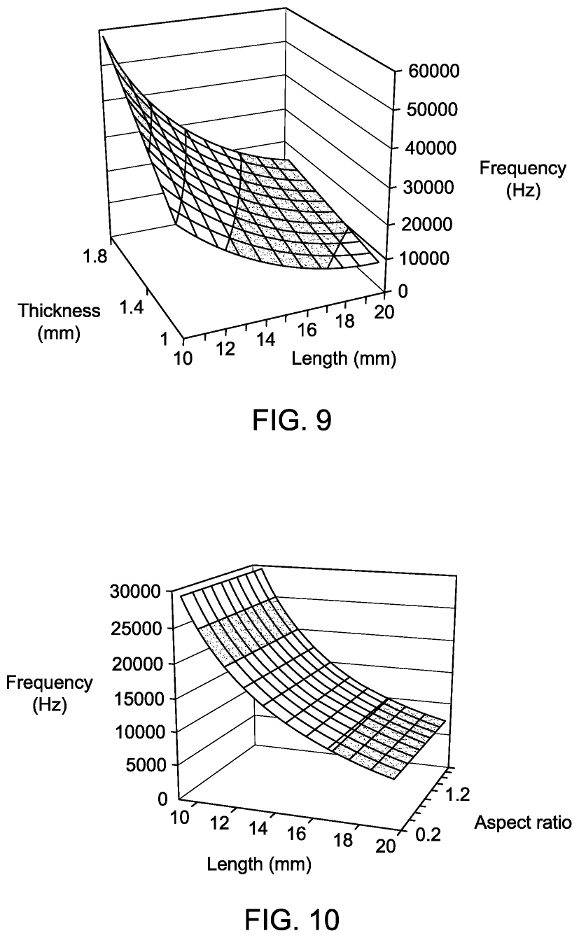

The formulas and properties provided above allow various characteristics of the blade 60 to be modeled, to apply to blades made from different materials. The modeling defines a range of combinations of blade thickness and length which give the blade the desired resonant frequency. FIG. 9 shows the frequency of a blade made of bulk diamond as a function of blade thickness and length. As shown in FIG. 10, unlike blades made from other materials, in the case of blades made from certain superhard materials (particularly those having low densities like diamond), the aspect ratio (ratio of blade length to blade width) has been found to have little impact on the resonant mode. In other words, the blade can be made narrow or wide, with little impact on the resonant frequency. This enables the blade to be made relatively wide without changing the length of the blade. This can provide the advantage of allowing increased flow rates to be handled by the apparatus by merely increasing the widths of the orifice and the blade, without changing the length, or thickness of the blade.

A process for mixing by producing shear and/or cavitation in a fluid is contemplated herein. The process utilizes an apparatus 20 such as that described herein. The process comprises providing a mixing or cavitation chamber, such as 22, and an element 30, such as orifice component system, with an orifice 32 therein. The element 30 comprises portions 44 surrounding the orifice 32, and the portions 44 surrounding the orifice 32 may be provided with a hardness in the hardness ranges specified above.

The process further comprises introducing at least one fluid into at least one entrance to the mixing or cavitation chamber 22 so that the fluid passes through the orifice 32 in the element 30. The at least one fluid can be supplied to the apparatus 20 in any suitable manner including, but not limited to through the use of pumps and motors powering the same. The pumps can supply at least one fluid to the apparatus under the desired pressure. There can be any suitable number of inlets, such as 26, to the cavitation chamber (e.g., 1, 2, 3, 4, 5, etc.) The fluid(s), or the mixture of the fluids, pass through the orifice 32 under pressure. The orifice 32 is configured, either alone, or in combination with some other component, to mix the fluids and/or produce cavitation in the fluid(s), or the mixture of the fluids.

### Antiperspirant Product

The antiperspirant composition may be from about 15% to about 50% solids, by weight of the antiperspirant composition, alternatively from about 20% to about 45%, alternatively from about 25% to about 40%, alternatively from about 28% to about 39%, and alternatively from about 30% to about 38%. The % solids, by weight of the composition, may be greater than about 20%, greater than about 25%, or greater than about 30%.

### Propellant

A spray device comprises a propellant stored in one or more reservoirs of the container. The propellant may be stored in the same reservoir as an antiperspirant composition or a separate reservoir, although it is preferred that the propellant is stored within the same reservoir as the antiperspirant composition to form the antiperspirant product. The propellant may be present in a liquefied form that is miscible with liquid carriers of the antiperspirant composition as well as gaseous state within a head space of the reservoir. The liquid propellant and the antiperspirant composition form a mixture that travels through the container, eventually exiting the container where the liquid propellant vaporizes to from a spray. The propellant may have a concentration from about 60% to about 90% or 95% or from about 70% to about 80% or from about 80% to about 90%, by weight of the antiperspirant product. Generally, as propellant concentration increases through these higher concentrations, the discharge may tend be more “gassy” possibly resulting in less deposition of the antiperspirant composition on the target surface as well as a wider spray pattern.

A wide variety of propellants may be used with the spray devices and antiperspirant compositions described herein. The spray device can be substantially free of compressed gas propellants such as nitrogen, air and carbon dioxide. Some suitable propellants may have a boiling point (at atmospheric pressure) within the range of from about −45° C. to about 5° C. Some suitable propellants may include chemically-inert hydrocarbons such as propane, n-butane, isobutane and cyclopropane, and mixtures thereof, as well as halogenaed hydrocarbons such as dichlorodifluoromethane (propellant 12) 1,1-dichloro-1,1,2,2-tetrafluoroethane (propellant 114), 1-chloro-1,1-difluoro-2,2-trifluoroethane (propellant 115), 1-chloro-1,1-difluoroethylene (propellant 142B), 1,1-difluoroethane (propellant 152A), dimethyl ether and monochlorodifluoromethane, and mixtures thereof. Some propellants suitable for use include, but are not limited to, A-46 (a mixture of isobutane, butane and propane), A-31 (isobutane), A-17 (n-butane), A-108 (propane), AP70 (a mixture of propane, isobutane and n-butane), AP40 (a mixture of propane, isobutene and n-butane), AP30 (a mixture of propane, isobutane and n-butane), HFO1234 (trans-1,3,3,3-tetrafluoropropene) and 152A (1,1 difluoroethane).

### Antiperspirant Compositions

### Viscosity

It may be desirable for the viscosity of the antiperspirant composition to be from about 200 centipoise (0.2 Pa·s), about 300 centipoise (0.3 Pa·s), about 500 centipoise (0.5 Pa·s), about 750 centipoise (0.75 Pa·s), about 1000 centipoise (1 Pa·s), about 2,000 centipoise (2 Pa·s), about 3,000 centipoise (3 Pa·s), about 4,000 centipoise (4 Pa·s), about 5000 centipoise (5 Pa·s), or about 7,000 centipoise (7 Pa·s), about 10,000 centipoise (10 Pa s), to about 50,000 centipoise (50 Pa·s), about 40,000 centipoise (40 Pa·s), or about 30,000 centipoise (30 Pa·s), or about 20,000 centipoise (20 Pa·s), or about 10,000 centipoise (10 Pa·s), or about 7,000 centipoise (7 Pa·s), about 5,000 centipoise (5 Pa·s) or about 4,000 centipoise (4 Pa·s) at 25° C.

An antiperspirant composition should be flowable so that it may be sprayed effectively from a spray device. Therefore, the antiperspirant composition may be devoid of sufficient concentrations and/or substantially free of ingredients that provide thickened stick or gel type of rheology in antiperspirant stick or gel products. Some common agents which may be excluded in sufficient amounts include hydrogenated castor oil, solid paraffins, silicone waxes, and mixtures thereof.

### Non-Volatile Silicone Fluids

The antiperspirant compositions may comprise one or more non-volatile silicone fluids. The non-volatile silicone fluid may function as the primary or principal liquid carrier for the antiperspirant active. As used herein, the term “non-volatile” refers to a material that has a boiling point above 250° C. (at atmospheric pressure) and/or a vapor pressure below 0.1 mm Hg at 25° C. Conversely, the term “volatile” refers to a material that has a boiling point less than 250° C. (at atmospheric pressure) and/or a vapor pressure about 0.1 mm Hg at 25° C. Incorporating a non-volatile silicone fluid in an antiperspirant composition may provide several benefits. First, non volatile silicone fluids can be more effectively deposited on the skin than volatile silicone fluids from aerosol antiperspirant compositions containing high levels of propellant, such as greater than 60% or 80% propellant. Deposition of high concentrations of a non-volatile carrier fluid in the antiperspirant composition is believed to reduce visible white residue at application, reduce visible white residue throughout the day and reduce antiperspirant composition transfer to clothes while dressing. Second, incorporating a non-volatile silicone fluid may increase the substantivity of the antiperspirant composition on skin, thereby potentially increasing antiperspirant efficacy, as the antiperspirant composition may form a film that more readily adheres to skin rather than flaking off or transferring to clothing throughout the day. Third, incorporating a non-volatile silicone fluid may also decrease the propensity for a visible residue to appear on skin (compared to using a volatile silicone fluid), as the non-volatile silicone fluid does not evaporate thereby leaving behind the white antiperspirant active as a visible residue. However, incorporating a non-volatile silicone fluid is not without potential tradeoffs. A perception of wetness post application (which may be undesirable for some consumers) is one tradeoff that may be associated with high concentrations of a non-volatile silicone fluid in an antiperspirant composition.

The total concentration of non-volatile silicones may be from about 30%, 35%, 40%, 45%, 50% to about 70%, 65%, 60%, 55% or 50% by weight of an antiperspirant composition. In some embodiments, the total concentration of non-volatile silicones may be from about 35% or 45% to about 55% by weight of an antiperspirant composition. The liquid materials of the antiperspirant composition may consist essentially of or primarily comprise a non-volatile, silicone fluid(s). Some non-volatile, silicone fluids that may be used include, but are not limited to, polyalkyl siloxanes, polyalkylaryl siloxanes, and polyether siloxane copolymers, and mixtures thereof. Some preferred non-volatile silicone fluids may be linear polyalkyl siloxanes, especially polydimethyl siloxanes (e.g., dimethicone). These siloxanes are available, for example, from Momentive Performance Materials, Inc. (Ohio, USA) under the tradename Element 14 PDMS (viscosity oil). Silicones Fluids from Dow Corning Corporation (Midland, Mich., USA) available under the trade name Dow Corning 200 Fluid series (e.g., 3 to 350 centistokes). Other non-volatile silicone fluids that can be used include polymethylphenylsiloxanes. These siloxanes are available, for example, from the General Electric Company as SF 1075 methyl phenyl fluid or from Dow Corning as 556 Fluid. A polyether siloxane copolymer that may be used is, for example, a dimethyl polyoxyalkylene ether copolymer fluid. Such copolymers are available, for example, from the General Electric Company as SF-1066 organosilicone surfactant. The non-volatile, silicone fluid may have an average viscosity from about 3 centistokes, 5 centistokes, 10 centistokes, 20 centistokes, or 50 centistokes to about 350 centistokes, 200 centistokes, 10⁰ centistokes, 50 or 30 centistokes at 25° C. (1 centistoke being equal to 1×10⁻⁶ m²/s). In some specific embodiments, the silicone fluid may have a viscosity from about 5 centistokes to about 10⁰ centistokes or 5 centistokes to about 50 centistokes or about 5 centistokes to about 30 centistokes. Higher viscosity non-volatile silicone fluids (e.g., greater than 100 centistokes or 200 centistokes or 350 centistokes) are preferably mixed with lower viscosity, non-volatile silicone fluids to achieve an appropriate antiperspirant composition viscosity in combination with the concentration of particulates. High viscosity, non-volatile silicone fluids (e.g., greater than 100, 200, or 350 centistokes) may comprise less than 25% by weight of an antiperspirant composition.

In some instances, the non-volatile silicone fluid is a polydimethylsiloxane fluid (also commonly referred to as dimethicone). It will be appreciated that a polydimethylsiloxane fluid may be further characterized by, optionally, its viscosity or its molecular weight or its formula or a combination thereof. In some instances, the polydimethylsiloxane fluid may have the characteristics described in, below.

TABLE 4

| | | |
|---|---|---|
| | Approximate | Approximate Average |
| | Molecular | Number of Monomer |
| Viscosity | Weight¹ | Units in the Polymer¹ |

| | | | |
|---|---|---|---|
| 5 | Centistokes | 800 | 9 |
| 10 | Centistokes | 1200 | 13 |
| 20 | Centistokes | 2000 | 27 |
| 30 | Centistokes | 2600 | 35 |
| 50 | Centistokes | 3800 | 50 |
| 100 | Centistokes | 6000 | 80 |
| 200 | Centistokes | 9400 | 125 |
| 350 | Centistokes | 13,700 | 185 |

¹The compositions of Examples herein, to the extent they contained a dimethicone fluid, were formulated utilitizing a Dow Corning DC200 series fluid, which is believed to have had average molecule weights and average number of monomer subunits falling within the approximate values of.

The polydimethylsiloxane fluid may have the following formula:

$$M - {D}_{X} - M$$

wherein M is (CH₃)₃SiO and D is 2CH₃(SiO) and X is equal to the average number of monomer units (see, e.g., Table 1) in the polymer minus 2. In some embodiments, X may be from about 6 to about 185, from about 9 to about 125, from about 9 to about 80, from about 9 to about 50, from about 13 to about 50 or from about 27 to about 50. In other embodiments, X may be from about 6 to about 35, from about 9 to about 35 or from about 13 to about 35. The term “approximate” as used in refers to ±10% of a given value.

While a wide variety of non-volatile silicone fluids or oils may be used in an antiperspirant composition, in some instances it may be desirable for the non-volatile silicone fluid(s) to consist essentially of or consist of or consist primarily of non-functionalized silicone fluids. In some embodiments, it may be further desirable for the non-volatile silicone fluid(s) to be substantially or completely free of non-functionalized siloxanes capable of reacting with the antiperspirant active via an acid-base reaction or a chelation reaction. This is in contrast to, for instance, U.S. Pat. No. 4,806,338 which proposes the use of functionalized siloxanes. Functionalized siloxanes may in some instances be disadvantageous in that they may react with the antiperspirant active, either via an acid-base reaction in the case of aminofunctional silicones, which are Lewis bases (the antiperspirant actives are Lewis acids), or via a chelation reaction (in the case of the carboxy functional silicones), which reactions can reduce the efficacy of the antiperspirant active. In addition, functional silicones of the type taught by U.S. Pat. No. 4,806,338 may have reduced solubility in the propellant (and vice versa) which may give rise to inhomogeneity in the product with resultant inhomogeneity of deposition on skin.

### Volatile Solvents

The compositions may include a volatile solvent or a mixture of volatile solvents. The volatile solvents may comprise from about 2%, from about 5%, from about 8%, from about 10%, from about 15%, from about 20%, to about 20%, to about 25%, to about 30%, to about 35%, to about 40%, to about 50%, about 60%, about 70%, about 80%, or any combination thereof, by weight of the composition, of volatile solvent. The volatile solvents useful herein may be relatively odorless and safe for use on human skin. Suitable volatile solvents may include C₁-C₄ alcohols and mixtures thereof. For example, ethanol may be used as the volatile solvent. Some other non-limiting examples of volatile solvents include methanol, propanol, isopropanol, butanol, and mixtures thereof.

### Nonvolatile Solvents

The composition may comprise from about 2%, from about 5%, from about 8%, from about 10%, from about 15%, from about 20%, to about 20%, to about 25%, to about 30%, to about 35%, to about 40%, to about 50%, to about 60%, to about 70%, to about 80%, or any combination thereof, by weight of the composition, of nonvolatile solvent. The composition may comprise a nonvolatile solvent or a mixture of nonvolatile solvents. Non-limiting examples of nonvolatile solvents include benzyl benzoate, diethyl phthalate, isopropyl myristate, propylene glycol, dipropylene glycol, triethyl citrate, and mixtures thereof. The composition may also be free of nonvolatile solvents.

### Liquid Fragrance Materials

An antiperspirant composition may also optionally comprise one or more liquid fragrance materials. Liquid fragrance materials are typically a mixture of perfume or aromatic components that are optionally mixed with a suitable solvent, diluent or carrier. Some suitable solvents, diluents or carriers for the perfume components may include ethanol, isopropanol, diethylene glycol monoethyl ether, dipropylene glycol, diethyl phthalate, triethyl citrate, isopropyl myristate and mixtures thereof. An antiperspirant composition may comprise from about 0.5%, 0.75%, 1%, 2%, 3% or 4% to about 10%, 8%, 6%, or 4%, 3% or 2% by weight of a liquid fragrance material.

The perfume component may be any natural or synthetic perfume component known to one skilled in the art of creating fragrances including, but not limited to, essential oils, citrus oils, absolutes, resinoids, resins, concretes, etc., and synthetic perfume components such as hydrocarbons, alcohols, aldehydes, ketones, ethers, acids, esters, acetals, ketals, nitriles, etc., including saturated and unsaturated compounds, aliphatic, carbocyclic and heterocyclic compounds. Some non-limiting examples of perfume components include: geraniol, geranyl acetate, linalool, linalyl acetate, tetrahydrolinalool, citronellol, citronellyl acetate, dihydromyrcenol, dihydromyrcenyl acetate, tetrahydromyrcenol, terpineol, terpinyl acetate, nopol, nopyl acetate, 2-phenylethanol, 2-phenylethyl acetate, benzyl alcohol, benzyl acetate, benzyl salicylate, benzyl benzoate, styrallyl acetate, amyl salicylate, dimethylbenzyl carbinol, trichloromethylphenyl-carbinyl acetate, p-tert.butyl-cyclohexyl acetate, isononyl acetate, vetiveryl acetate, vetiverol, alpha-n-amylcinammic aidehyde, alpha-hexylcinammic aldehyde, 2-methyl-3-(p-tert.butylphenyl)-propanol, 2-methyl-3-(p-isopropylphenyl)-propanal, 3-(p-tert.butylphenyl)-propanal, tricyclodecenyl acetate, tricyclodecenyl propionate, 4-(4-hydroxy-4-methylpentyl)-3-cyclohexene carbaldehyde, 4-(4-methyl-3-pentenyl)-3-cyclohexene carbaldehyde, 4-acetoxy-3-pentyltetrahydropyran, methyldihydrojasmonate, 2-n-heptylcyclopentanone, 3-methyl-2-pentylcyclopentanone, n-decanal, 9-decenol-1, phenoxyethyl isobutyrate, phenyl-acetaldehyde dimethyl acetal, phenylacetaldehyde diethyl acetal, geranonitrile, citronellonitrile, cedryl acetate, 3-isocamphylcyclohexanol, cedryl methyl ether, isolongifolanone, aubepine nitrile, aubepine, heliotropine, coumarin, eugenol, vanillin, diphenyl oxide, hydroxycitronellal, ionones, methylionones, isomethylionones, irones, cis-3-hexenol and esters thereof, indane musk fragrances, tetralin musk fragrances, isochroman musk fragrances, macrocyclic ketones, macrolactone musk frangrances, ethylene brassylate, aromatic nitro-musk fragrances. Some perfume components are also described in Arctander, Perfume and Flavour Chemicals (Chemicals), Vol. I and II (1969) and Arctander, Perfume and Flavour Materials of Natural Origin (1960).

### Other Liquid Materials

Other liquid materials may be optionally included in an antiperspirant composition. The liquid materials of the antiperspirant composition may comprise less than 30%, 20%, 10%, or less than 5% by weight of liquid materials other than non-volatile, silicone fluids. Said in another way, the liquid materials of the antiperspirant composition may comprise more than 70%, 75%, 80%, 85%, 90% or about 100% by weight of non-volatile silicone fluids.

An antiperspirant composition may comprise less than 10%, 5%, 1%, or 0.5% by weight of a volatile silicone fluid. An antiperspirant composition may be substantially or completely free of a volatile silicone fluid.

An antiperspirant composition may optionally comprise one or more silicone gums. The term “gum” is used to refer to a material that has a viscosity within the range from about 100,000 to about 100 million centistokes at 25° C. and which is slowly flowable, as opposed to a rigid solid, which is not flowable, or a liquid, which is too flowable. Silicone gum materials are blends of a silicone gum and a diluents, wherein the diluents reduces the viscosity of the blend. Some common diluents can include but are not limited to 5 centistoke dimethicone, 50 centistoke dimethicone, 100 centistoke diemthicone or cyclopentasiloxane. In some embodiments, the antiperspirant composition may be substantially or completely free of cyclopentasiloxane. The silicone gum may comprise high viscosity polydimethylsiloxanes with terminal methyl (e.g., dimethicone) or hydroxyl (e.g., dimethiconol) groups. Silicone gums may have a molecular weight from 100,000 Daltons greater than 2,000,000 Daltons. The viscosity of the silicone gums (without a diluents) may range from 300,000 centistokes to greater than 2,500,000 centistokes or higher compared to the viscosity of silicone gum materials (inclusive of diluents) which may be less than 10,000 centistokes. Some examples of silicone gums and silicone gums materials include, but are not limited to, quaternary ammonium functional silicones such as DC7-6030 available from Dow Corning and 34720, 34749, 34731, 33134, SF-96, SF-1066, SF18 (350), SE30 and SE32 available from General Electric.

A silicone gum (or silicone gum material) may be added to an antiperspirant composition to further increase deposition and/or substantivity of the antiperspirant composition and/or increase the drop size of the aerosol spray particles. The improvement in deposition can be illustrated by evaluating the deposition of a test sample comprising 85% A46 propellant, 14.64% 50 centistoke dimethicone, and 0.36% DC1503 (note this is made by mixing 97% 50 cst dimethicone with 3% DC1503, which contains 12% silicone gum, and then adding that mixture to the propellant at a 15%). Deposition testing of this sample using the same valve and actuator as the aforementioned samples showed a deposition efficiency of about 58%. This represents a 38% improvement in deposition versus the aforementioned test sample comprising only 50 cst dimethicone and a more than 100% improvement over the sample comprising only cyclopentasiloxane. Maximizing liquid deposition in the test sample comprising a high concentration of a non-volatile silicone fluid and a high propellant concentration is desirable not only to reduce visible white but also to reduce potential inhalation hazards. Volatile silicones, such as cyclopentasiloxane, may be removed from the lung via exhaling while nonvolatile materials are less likely to be removed by the mechanism. As such it is desirable to limit inhalable non-volatile silicone materials by increasing the deposition efficiency via the addition of silicone gum.

However, formulating an antiperspirant composition with a silicone gum in combination with relatively high concentrations of a non-volatile silicone fluid and/or relatively high concentrations of total particulates may involve a number of tradeoffs. For example, too much of a silicone gum may dramatically increase viscosity of the antiperspirant composition and the risk of clogging of the container actuator and/or valve, particularly where there is already a relatively high concentration of total particulates. Still further, too much of a silicone gum may reduce the diameter of the spray making it more difficult for a user to achieve complete coverage of an axillia (typically a 7.5 cm×12.5 cm area) during application as well as potentially creating regions of high antiperspirant composition dosage, thereby potentially impacting skin feel. Further, some silicone gums, such as the quaternary ammonium functional silicones described in U.S. Pat. No. 7,815,899 may have an undesirable odor (e.g., a fish-like odor) associated therewith, which may then be imparted to an antiperspirant composition in some instances.

Generally, it is believed that the concentration of the silicone gum may be increased as propellant concentration increases, all other variables being equal. Conversely, it is believed that as the amount of particulates increases, the concentration of the silicone gum should be decreased as the amount of particulates increases, all other variables being equal. This is believed particularly true within the particulate range of 40% to 60% by weight of the antiperspirant composition, as mounding of the antiperspirant composition may result.

Given the one or more potential challenges associated with incorporating a gum and more particularly a silicone gum, an antiperspirant composition may have a concentration of a silicone gum from about 0.1%, 0.3%, 0.4%, 0.5%, 0.6%, 0.7% or 0.8% to about 1.5%, 1.25%, 1%, 0.9%, 0.8%, 0.7%, 0.65, 0.5%, or 0.4% by weight of the antiperspirant composition. In some instances, the most preferred concentration of silicone gum by weight of the antiperspirant composition is from about 0.3% to about 0.8% in order to balance pattern diameter/quality with deposition. In some instances, the antiperspirant composition may have from about 0.1% to about 0.6% of a silicone gum when paired with a propellant concentration from 70% to 80% and a particulate concentration from 40% to 50%. In some instances, the antiperspirant composition may have from about 0.1% to about 0.4% of a silicone gum when paired with a propellant concentration from 70% to 80% and a particulate concentration from 50% to 60%. In some instances, the antiperspirant composition may have from about 0.3% to about 1.5% of a silicone gum when paired with a propellant concentration from 80% to 90% and a particulate concentration from 40% to 50%. In some instances, the antiperspirant composition may have from about 0.3% to about 1% of a silicone gum when paired with a propellant concentration from 80% to 90% and a particulate concentration from 50% to 60%. While it is believed to be very desirable to include a silicone gum in an antiperspirant composition comprising a non-volatile silicone fluid and at propellant concentrations from about 70% to about 90% or even about 95%, it is also contemplated that in some instances it may be desirable for the antiperspirant composition to be substantially or completely free of a silicone gum.

If a silicone gum is included, any silicone gum having a viscosity within the ranges described herein may be used, provided it is soluble in the liquid carrier, propellant or a combination thereof of the antiperspirant composition. Some suitable silicone gums include silicone polymers of the dimethyl polysiloxane type, which may have other groups attached, such as phenyl, vinyl, cyano, or acrylic, but the methyl groups should be in a major proportion. Silicone polymers having a viscosity below about 100,000 centistokes (molecular weight below about 100,000) at 25° C. are not considered silicone gums here but are rather, typically, considered a silicone fluid. One non-limiting example of silicone gum suitable for use is a silicone/gum fluid blend comprising a dimethiconol gum having a molecular weight form about 200,000 to 4,000,000 along with a silicone fluid carrier with a viscosity from about 0.65 to 100 mm² s⁻¹. An example of this silicone/gum blend is available from Dow Corning, Corp. of Michigan, USA under the trade name DC-1503 Fluid (88% dimethicone fluid/12% dimethiconol). Other silicone gums materials include SF1236 Dimethicone, SF1276 Dimethicone, and CF1251 Dimethicone available from Momentive Performance Materials, Inc. of NY, USA.

An antiperspirant composition is preferably substantially or completely free of water added as separate ingredient (i.e., anhydrous), as too much added water may result in several deleterious effects such as: 1) increasing the propensity for antiperspirant active particulates to agglomerate (thereby increasing the propensity for clogging), and 2) reducing dry feel on skin. It will be appreciated that even an anhydrous antiperspirant composition may still contain some water that is bound with an ingredient (e.g., antiperspirant active, tapioca material, etc.) otherwise added to the antiperspirant composition.

### Particulate Materials

Delivering a sufficient concentration of particulates to the skin is believed to improve the skin feel of an antiperspirant composition comprising a high concentration of a non-volatile silicone fluid. It is believed that an antiperspirant composition comprising a total non-volatile liquid material to total particulate material ratio (L/P ratio) from about 0.6, 0.8, 1, 1.2, or 1.4 to about 1.6, 1.4, 1.2 or 1 may balance the tradeoff between enough particulates to provide acceptable skin feel while minimizing the appearance of residue. An antiperspirant composition may have a total particulate concentration from about 30%, 35%, or 40% to about 50% or 45% by weight of the antiperspirant composition.

The antiperspirant composition may comprise a variety of particulate materials. However, it is believed that the type (e.g., hydrophilic v. hydrophobic) and concentrations of particulate materials included in an antiperspirant composition may, in some instances, impact skin feel, release of the antiperspirant active, and the propensity for clogging in the spray device. For example, too much antiperspirant active may result in a wet or sticky skin feel due to the propensity of antiperspirant actives to become sticky when hydrated (e.g., by perspiration) even within the L/P ratios previously described. In addition, too much of a hydrophobic particulate material may reduce release of the antiperspirant active from the composition. Conversely, inclusion of a hydrophilic particulate material may advantageously aid release of the antiperspirant active, which may be beneficial in a composition comprising a high concentration of a non-volatile silicone fluid.

However, hydrophilic materials may increase the risk of clogging in the presence of water. Therefore, it may be desirable to balance these and other design considerations when incorporating particulate materials in an antiperspirant composition comprising a non-volatile silicone fluid. It is believed that L/P ratios from about 1 to about 1.6 may be particularly beneficial in some instances for balancing the tradeoff between skin feel and residue in an antiperspirant composition comprising a non-volatile silicone fluid.

Some examples of particulate materials suitable for use include, but are not limited to, antiperspirant actives, powders (e.g., starch materials), encapsulated fragrance materials and bulking or suspending agents (e.g., silicas or clay materials). Other types of particulates may also be incorporated in an antiperspirant composition.

### Antiperspirant Actives

An antiperspirant composition can comprise one or more antiperspirant actives. The antiperspirant actives are in a particulate form (rather than being solubilized) in the antiperspirant composition. Therefore, it may be desirable that the antiperspirant composition is provided in a form other than an emulsion or is substantially or completely free of solubilizers for the antiperspirant active. The antiperspirant composition may be provided in the form of a liquid dispersion (including suspensions and colloids). This is in contrast to, for instance, WO 03/002082 which discloses solubilizing the antiperspirant active in an emulsion having a disperse phase and a continuous phase.

The compositions described herein may be free of, substantially free of, or may include an antiperspirant active (i.e. any substance, mixture, or other material having antiperspirant activity). The antiperspirant active may be any particle having antiperspirant activity. The antiperspirant active is preferably insoluble in the liquid components of the antiperspirant composition. Since the amount of antiperspirant active may significantly impact skin feel, an antiperspirant composition may comprise from about 14% 16%, 18%, 20%, 22%, or 24% to about 38%, 36%, 34%, 32%, 30%, 28%, or 26% by weight of a particulate antiperspirant active. In some instances, it may be desirable to utilize a low concentration of the antiperspirant active, such as less than 20% or 18% by weight of the antiperspirant composition. The antiperspirant active concentrations refer to the anhydrous amount that is added.

Some examples of suitable antiperspirant actives include astringent metallic salts, particularly including the inorganic and organic salts of aluminum. Some non-limiting examples exemplary aluminum salts that can be used include aluminum chloride and the aluminum hydroxyhalides having the general formula Al₂(OH)ₐQ_{b}XH₂0 where Q is chloride, bromide, or iodide (preferably chloride), a is from about 2 to about 5, and a+b=about 6, and a and b do not need to be integers, and where X is from about 1 to about 6, and X does not need to be an integer. Particularly preferred are the aluminum chlorhydroxides referred to as “5/6 basic chlorhydroxide” wherein “a” is 5 and “2/3 basic chlorhydroxide” wherein “a” is 4. Aluminum salts of this type can be prepared in the manner described more fully in U.S. Pat. Nos. 3,887,692; 3,904,741; and 4,359,456. Preferred compounds include the 5/6 basic aluminum salts of the empirical formula A1₂(OH)₅DI₂H₂0; mixtures of AIC1₃6H₂0 and A1₂(OH)₅CI₂H₂O with aluminum chloride to aluminum hydroxychloride weight ratios of up to about 0.5. The antiperspirant active may be, for example, aluminum cholorohydrate.

The aluminum salt may be prepared by methods well known in the art. In some embodiments, the aluminum salts may be made by applying heat to a dilute aqueous solution of an aluminum salt (e.g., less than 20% of an aluminum salt by weight of the dilute solution) to form a solid aluminum salt comprising aluminum hydrolysis polymers. Some non-limiting examples of such methods are described in U.S. Pat. Nos. 4,871,525 and 4,359,456.

### Substantially Inert Particulate Materials

The balance of the total particulate concentration of an antiperspirant composition may comprise excipient particulate materials that are substantially inert with respect to itself and/or antiperspirant active, meaning there are no significant particle to particle interactions with respect to itself and/or the antiperspirant active when present in the antiperspirant composition. Excipient particulate materials exclude clays and silicas added to an antiperspirant composition as bulking or suspending agents, as these particles can exhibit strong particle to particle interactions. The excipient particulate materials may be either hydrophilic or hydrophobic (including hydrophobically modified, which tend to be moderately hydrophobic). Some non-limiting examples of substantially inert excipient particulate materials that may be included in an antiperspirant composition include encapsulated fragrance materials; native starches such as tapioca, corn, oat, potato, and wheat starch particulates or hydrophibically modified versions of these starches; talc; calcium carbonate; perlite; mica and polyethylene beads. One non-limiting example of a hydrohobically modified corn starch material suitable for use comprises aluminum starch octenylsuccinate, which is available under the trade name Dry Flo PC or Dry Flo Pure from Akzo Nobel, Netherlands. The substantially inert particulates may be free flowing. An antiperspirant composition may comprise from about 0.25%, 0.5%, 1%, 5%, 10%, 12%, or 14% to about 25%, 22%, 20%, 18%, or 16% by weight of the antiperspirant composition of substantially inert particulates. One substantially inert particulate material believed to be suitable for use is a hydrophilic or hydrophobically modified tapioca starch material. A tapioca starch material may be particularly beneficial as it is unlikely to induce an allergic reaction if inhaled. Tapioca is a starch which may be extracted from the cassava plant, typically from the root, which may then be processed or modified as known in the art. Tapioca starches are, advantageously, substantially non-allergenic. One non-limiting example of a hydrophobically modified tapioca starch material suitable for use comprises a silicone grafted tapioca starch, which is available under the trade name Dry Flo TS from AkzoNobel of the Netherlands. The INCI name is tapioca starch polymethylsilsesquioxane and may be produced by a reaction of methyl sodium siliconate (polymethylsilsesquioxane) and tapioca starch. This silicone grafted tapioca starch material is commercially available as CAS No. 68989 December 8. The silicone grafted tapioca starch material can be formed using any known means, including, but not limited to those methods described in U.S. Pat. Nos. 7,375,214, 7,799,909, 6,037,466, 2,852,404, 5,672,699, and 5,776,476. Other non-limiting examples of hydrophobically modified tapioca starch materials that are suitable for use include Dry Flo AF (silicone modified starch from Akzo Nobel), Rheoplus PC 541 (Siam Modified Starch), Acistar RT starch (available from Cargill) and Lorenz 325, Lorenz 326, and Lorenz 810 (available from Lorenz of Brazil). In some specific embodiments, the tapioca starch material may be hydrophilic in order to facilitate release of the antiperspirant active during use. One non-limiting example of a hydrophilic tapioca starch material suitable for use is available under the trade name Tapioca Pure available from Akzo Nobel. In some specific embodiments, the substantially inert particulate material comprises a hydrophilic tapioca material, a hydrophobic tapioca material or a mixture thereof.

An antiperspirant composition may optionally comprise one or more particulate fragrance carriers or materials that may or may not encapsulate a perfume component. Fragrance carriers are typically particulates, which would be considered part of the total particulate concentration of the antiperspirant composition. The fragrance carriers are preferably hydrophobic in order to minimize particle-to-particle interactions. The fragrance carriers may be either full or empty. A full fragrance carrier is a fragrance carrier that encapsulates or otherwise contains a perfume component while the fragrance carrier is stored within the spray device. Full fragrance carriers may release their perfume components by a variety of mechanisms post delivery from the spray device to provide a desired aroma or fragrance experience for a user. For example, the perfume components may be released by moisture upon wetting of the fragrance carrier, e.g., by perspiration or other body fluids. Alternatively or in addition thereto, the perfume components may be released by fracture of the carrier, such as by the application of pressure or a shearing force. An empty fragrance carrier is a fragrance carrier that does not contain a perfume component while stored within the spray device. One non-limiting example of an empty fragrance carrier is an uncomplexed cyclodextrin material.

Some non-limiting examples of fragrance carriers suitable for encapsulating a perfume component include, but are not limited to, oligosaccharides (e.g., cyclodextrins), starches, polyethylenes, polayamides, polystyrenes, polyisoprenes, polycarbonates, polyesters, polyacrylates, vinyl polymers, silicas, and aluminosilicates. Some examples of fragrance carriers are described in USPNs 2010/0104611; 2010/0104613; 2010/0104612; 2011/0269658; 2011/0269657; 2011/0268802; U.S. Pat. Nos. 5,861,144; 5,711,941; 8,147,808; and 5,861,144.

An antiperspirant composition may comprise from about 0.25%, 0.5%, 0.75%, 1%, or 2% to about 20%, 16%, 12%, 10%, 8%, 6% or 4% by weight of the antiperspirant composition of fragrance carriers. In some instances, the substantially inert excipient particles of the antiperspirant composition consist essentially of or completely of full fragrance carriers, empty fragrance carriers, or mixtures thereof. An antiperspirant composition may comprise from about 0.25%, 0.5%, 0.75%, or 1% to about 6%, 4% or 2% by weight of the antiperspirant composition of full fragrance carriers. An antiperspirant composition may comprise from about 0.25%, 0.5%, 1%, or 2% to about 16%, 12%, 10%, 8%, 6% or 4% by weight of the antiperspirant composition of empty fragrance carriers.

In some instances, it may be desirable to incorporate a mixture of empty fragrance carriers and full fragrance carriers in the antiperspirant composition, wherein the empty fragrance carriers may be included to achieve the desired overall particulate concentration without the risk of perfume over-dosing.

In some instances, it may be desirable to provide a mixture of fragrance carriers and native starch powders to achieve the desired particle concentration. For example, from about a 20:80 to 80:20 (fragrance carrier to starch) mixture might be utilized. In some instances, a 50:50 mixture might be utilized and in other instances the native starch powders might have a concentration equal to about or less than 6% by weight of the antiperspirant composition while the concentration of the fragrance carriers might be equal to about or less than 9% by weight of the antiperspirant composition.

A wide variety of perfume components may be used with the fragrance carriers, including but not limited to volatile perfume components having a boiling point at normal pressure of less than about 260° C., more preferably less than about 250° C., and perfume components having significant low odor detection threshold, and mixtures thereof. The boiling points of many perfume components are given in, e.g., “Perfume and Flavor Chemicals (Aroma Chemicals),” Steffen Arctander, published by the author, 1969.

### Bulking and Suspending Agents

An antiperspirant composition may comprise a bulking or suspending agent. In some instances, it may be desirable to include a bulking or suspending agent in the antiperspirant composition in order to reduce the risk of caking of the antiperspirant composition at the bottom of the container and/or to aid in the redispersion of the antiperspirant composition upon shaking without significant clumping so as to reduce the risk of clogging any small orifices within the spray device. This may be particularly useful as antiperspirant actives are dense and tend to settle quickly and/or may be prone to caking in the presence of moisture. Significant settling and/or agglomeration of particulates in an antiperspirant composition may complicate delivery of a uniform dose of the antiperspirant active from a spray device. This in turn may negatively impact skin feel or contribute to the appearance of a white residue. While other solutions for addressing redispersion, settling and/or caking may be employed, there may also be tradeoffs involved. For example, U.S. Pat. No. 7,815,899 suggests utilizing a high viscosity polymeric material (e.g., a quaternary ammonium functional silicone) to reduce the settling rate. However, this approach may, in some instances, have tradeoffs. For example, some quaternary silicones have a strong odor from amine impurities that can interfere with fragrance of the product. Moreover, these amines may negatively interact with the active via a lewis acid/base reaction.

The bulking or suspending agent may be hydrophobic, hydrophilic or comprise mixtures thereof. In some specific embodiments, these materials may be hydrophilic in order to facilitate release of the antiperspirant active during use. Some examples of silica materials that may be used include, but are not limited to, colloidal silicas. Some non-limiting examples of silica materials are available from Evonik Industries under the trade names Aerosil 200SP, Aerosil 300SP and Aerosil R972.

In some instances, the antiperspirant composition may include a clay material. Some non-limiting examples of clay materials include montmorillonite clays and hydrophobically treated montmorillonite clays. Montmorillonite clays are those which contain the mineral montmorillonite and may be characterized by a having a suspending lattice. Some examples of these clays include but are not limited to bentonites, hectorites, and colloidal magnesium aluminum silicates. Some non-limiting examples of organoclays include modified bentonite, modified hectorite, modified montorlinite and combinations thereof, some examples of which are available under the trade names Bentone 27 (stearalkonium bentonite), Bentone 34 (stearalkonium bentonite) and Bentone 38 (disteardimonium hectorite) from Elementis Specialities Plc. and Tixogel VPV (quaternium 90-bentonite), Tixogel VZV (stearalkonium bentonite), Tixogel LGM (stearalkonium bentonite) and Claytone SO (stearalkonium bentonite) from Southern Clay Products.

The antiperspirant composition may also comprise a clay activator, such as propylene carbonate, triethyl citrate, methanol, ethanol, acetone, water and mixtures and derivatives thereof. Clay activators may also strongly interact with an antiperspirant active (e.g., leading to clumping or coating of the antiperspirant active and/or changes in active polymer structure which may reduce antiperspirant efficacy). Therefore, it may be desirable to limit the amount of clay activator present in the antiperspirant composition to between about 0.5%, 0.75%, 1%, 1.25%, or 1.5% to about 3%, 2%, or 1.75% by weight of the antiperspirant composition.

### Spray Devices

To avoid over-dosing of the antiperspirant composition, it is desirable that the spray device have a total mass flow rate of the propellant/antiperspirant composition mixture of less than 1.25 grams/sec or from about 0.5 grams/sec to about 1.3 grams/sec, or from about 0.6 grams/sec to about 1.0 grams/sec, or from about 0.7 grams/sec to about 1.0 grams/sec. The spray device may have an antiperspirant composition mass flow rate less than 0.3 grams/sec or from about 0.1 grams/sec to about 0.3 grams/sec or from about 0.1 grams/sec to 0.2 grams/sec or from about 0.15 grams/sec to about 0.2 grams/sec. It is believed that mass flow rates greater than described above may lead to a wet or sticky skin feel because the total amount of antiperspirant composition deposited on the skin may be too great.

The amount of antiperspirant active delivered to a target surface by a two second application from a spray device may be from about 40 mg, 50 mg, 60 mg, or 70 mg to about 100 mg, 90 mg, or 80 mg. The total amount of antiperspirant composition delivered to a target surface by a two second application of a spray device may be from about 0.1 grams to about 0.4 grams, or from about 0.2 grams to about 0.4 grams, or from about 0.2 grams to about 0.3 grams. The amount of liquid fragrance material delivered to a target surface by a two second application of a spray device may be from about 3 mg to about 20 mg, or from about 6 mg to about 15 mg, or from about 6 mg to about 12 mg. The amount of full fragrance carriers delivered to a target surface by a two second application of a spray device may be from about 0.75 mg to about 15 mg, or from about 1 mg to about 12 mg, or from about 1 mg to about 9 mg. The spray device may have a deposition efficiency, of either the antiperspirant composition and/or the antiperspirant active and/or the liquid fragrance material, that is from about 50%, 55%, 60%, 70% or 75% to about 85%, 80%, or 75%.

One example of a non-limiting valve assembly suitable for use is described in U.S. Pat. No. 4,396,152. One example of a valve assembly is available from the Precision Valve Company (USA) under the tradename Ecosol.

A user of a spray device may initiate a spray by depressing an actuator, thereby opening a valve which enables a liquid propellant/antiperspirant composition mixture to exit the actuator. Prior to actuation, it may be desirable to shake or rotate the product to redisperse the liquid and particulate materials. While usage time can vary widely, users may depress the actuator from about 2 seconds to about 5 seconds, or from about 2 seconds to about 4 seconds, or from about 2 seconds to about 3 seconds to provide a burst of antiperspirant composition for deposition to an underarm or axillia skin surface. A spray device may be sized to provide a total spray time from about 60 seconds to about 200 seconds, or from about 70 seconds to about 150 seconds, for from about 90 seconds to about 130 seconds, thereby providing from about 15 to about 50 two second uses before exhaustion.

The term “antiperspirant composition” refers to any composition containing an antiperspirant active and which is intended to be sprayed onto skin, exclusive of a propellant.

The term “antiperspirant efficacy” refers to the amount of wetness protection provided by application of an antiperspirant composition to an underarm area (or axillia) by a spray device. Antiperspirant efficacy may be quantified by the amount (mg) of sweat collected following exposure to a hot room compared to a baseline amount.

The term “bulking or suspending material” refers to a material which is intended to reduce settling of a particulate from a liquid and/or reduce the severity of particulate caking post settling.

The term “deposition efficiency” refers to the percentage of a material (e.g., antiperspirant active, fragrance material, antiperspirant composition, etc.) that is deposited on a target surface compared to the amount of material that exits a spray device.

The term “particulate” refers to a material that is solid or hollow or porous (or a combination thereof) and which is substantially or completely insoluble in the liquid materials of an antiperspirant composition.

The term “propellant” refers to one or more gases that are used to pressurize the antiperspirant composition to facilitate egress of the antiperspirant composition from the container. Some propellants may be a mixture of gases (e.g., A-46 which may be a mixture of isobutane, butane and propane). A propellant may be in the form of a liquid (i.e., a liquefied gas) when under pressure within the reservoir of a spray device. In addition, a propellant may be in its gaseous state within the head space of the reservoir. A propellant may be present in both a liquefied form and its gaseous state within the reservoir. Unless specified otherwise (e.g., liquid propellant or gaseous propellant), the term propellant is intended to encompass the liquefied form and the gaseous state individually and collectively.

The term “substantially free of” refers to an amount of a material that is less than 1%, 0.5%, 0.25%, 0.1%, 0.05%, 0.01%, or 0.001% by weight of an antiperspirant composition. “Free of” refers to no detectable amount of the stated material, ingredient or thing.

The term “total fill” refers to the total amount of materials added to or stored within a reservoir(s) of a container. For example, total fill includes the propellant and antiperspirant composition stored within a spray device after completion of filling and prior to first use.

The term “viscosity” means dynamic viscosity (measured in centipoise, cPs, or Pascal-second, Pa·s) or kinematic viscosity (measured in centistokes, cSt, or m²/s) of a liquid at approximately 25° C. and ambient conditions. Dynamic viscosity may be measured using a rotational viscometer, such as a Brookfield Dial Reading Viscometer Model 1-2 RVT available from Brookfield Engineering Laboratories (USA) or other substitutable model as known in the art. Typical Brookfield spindles which may be used include, without limitation, RV-7 at a spindle speed of 20 rpm, recognizing that the exact spindle may be selected as needed by one skilled in the art. Kinematic viscosity may be determined by dividing dynamic viscosity by the density of the liquid (at 25° C. and ambient conditions), as known in the art.

All percentages are by weight of the cosmetic composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 21° C. and at ambient conditions, where “ambient conditions” means conditions under about 1 atmosphere of pressure and at about 50% relative humidity. All weights as they pertain to listed ingredients are based on the active level and do not include carriers or by-products that may be included in commercially available materials, unless otherwise specified. All numeric ranges are inclusive of narrower ranges; delineated upper and lower range limits are interchangeable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

### EXAMPLES

The following data and examples are provided to help illustrate the skincare compositions described herein. The exemplified compositions are given solely for the purpose of illustration and are not to be construed as limitations of the present disclosure, as many variations thereof are possible without departing from the spirit and scope of the disclosure. All parts, percentages, and ratios herein are by weight unless otherwise specified.

TABLE 5
Example (AP Neuter)

| Common/Trade name | Wt. % |
|---|---|
| Dimethicone, 5 cst | Q.S. |
| Aluminum Chlorohydrate | 20-30 |
| C12-15 Alkyl Benzoate | 0-9 |
| Isopropyl Myristate | 0-9 |
| PPG-14 Butyl Ether | 0-8 |
| Dimethicone (and) Dimethiconol | 0-5 |
| Disteardimonium Hectorite | 2-5 |
| Propylene Carbonate | 0.3-1.7 |
| Encapsulated Fragrance | 0-2 |
| Minors and Additional Fragrance | 0-4 |

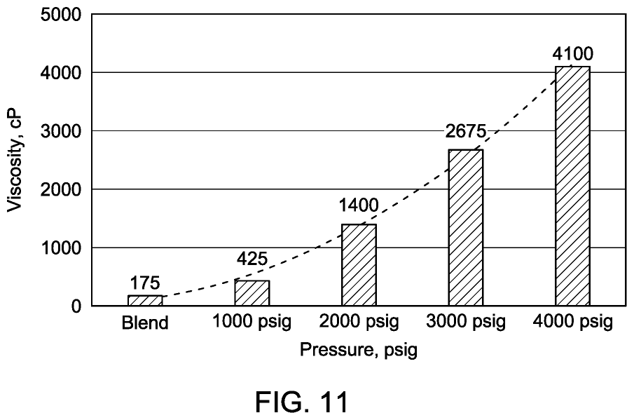

FIG. 11 shows the viscosity of the Example in made following the method described in FIG. 1 and accompanying text. For the examples at 1000-4000 psig, the blended mixture was pumped through an apparatus featuring an orifice size of 0.0007 square inches at a pressure of 1000, 2000, 3000, and 4000 psig and the viscosity was measured as described hereafter.

As shown in FIG. 11, the blend is the Example AP Neuter without any pressure. FIG. 11 shows that as the pressure increases, the viscosity of the composition increases. This indicates that more structure is formed with higher pressure, and it can be advantageous to use a pressure of from about 1000 psig (6895 kPa) to about 5000 psig (34,474 kPa), alternatively a pressure greater than 3000 psig (20,684 kPa), and alternatively a pressure greater than 4000 psig (27,579 kPa).

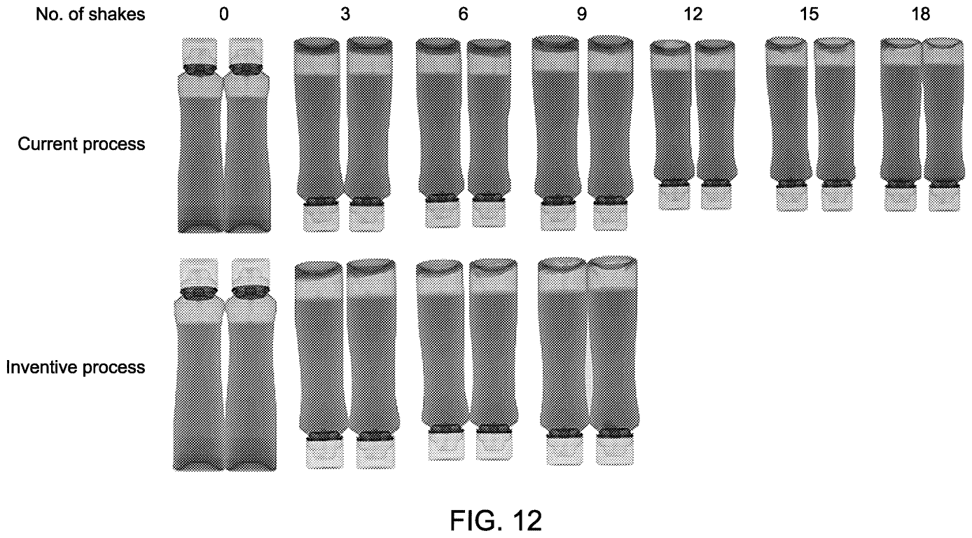

FIG. 12 shows x-ray pictures of aerosol products containing the Example in. The aerosol products from the current process were made as follows:
1. Combine Dimethicone (5 cst), Isopropyl Myristate, C12-C15 Alkyl Benzoate, and Mineral Oil and mix well thoroughly.
2. Add clay to the mixture, mix well, and then mill using any suitable milling device, such as a DR2000/5 mill available from IKA.
3. Incorporate propylene carbonate, mix, and mill again.
4. Add Dimethicone, Dimethiconol, and PPG-14 Butyl Ether, then mix thoroughly.
5. Introduce Aluminum Chlorohydrate and Envicap, mix well, and mill with the IKA mill.
6. Pump the mixture into a storage tank and maintain agitation until ready for packaging to create the AP neuter.
7. Transfer the product from the storage tank to the filler and gasser for final packaging and addition of perfume and propellant.

The aerosol products using the inventive process were made following the method described in FIG. 1 and accompanying text. The blended mixture was pumped through an apparatus featuring an orifice size of 0.0007 square inches at a pressure of 4000 psig (27,579 kPa).

For both the current process and the inventive process, the AP neuter was combined with 2% perfume and 60% propellant to form the aerosol AP product. The propellant was a mixture made up of 66% A17 propellant, which is primarily a hydrocarbon blend containing over 95% n-butane and less than 5% isobutane, along with 34% Hydrofluorocarbon 152A.

After a ten-week storage period at ambient conditions, the aerosol products were tested. As shown in FIG. 12, x-ray images revealed the presence of compacted cakes at the bottom of the can, if present. As shown in FIG. 12, both the current process and the inventive process produce compacted cakes in the bottom of the can without shaking. However, after only three shakes, there is significantly less compacted cake in the bottom of the can for the inventive process, as compared to the current process. For the inventive process, the product was substantially homogeneous after 9 shakes, which is substantially less than the 18 shakes that are required to get a homogeneous composition with the current product. This indicates that although the cake was initially present, it wasn't as dense and compacted as compared to the current process. Thus, the inventive process forms a composition that is more consumer acceptable.

It was also found that using the dual-stream process, as shown in FIG. 2, yields an AP product comparable to those made by the current process and shown in FIG. 12.

### Combinations

A. A method of making an antiperspirant composition, the method comprising:
   a. combining a first group of components comprising an antiperspirant active, a hydrophobically-modified clay, an anhydrous solvent, and a clay activator in a stream;
   b. transferring the stream through an apparatus for mixing liquids by producing shear and cavitation, said apparatus comprising:
      i. a mixing chamber, said mixing chamber comprising an entrance, at least one inlet, and an outlet; and
      ii. an element with an orifice therein, said element being located adjacent the entrance of said mixing chamber, wherein said orifice is configured to produce shear or cavitation in the second stream, wherein the pressure at the orifice is greater than 500 psig;
   wherein the apparatus increases the viscosity of the stream forming the antiperspirant neuter;
   c. adding one or more late product differentiation materials to form the antiperspirant composition.
B. The method according to paragraph A, wherein the size of the orifice is from about 0.0007 in² (0.45 mm²) to about 0.02 in² (12.9 mm²), preferably the size of the orifice is from about 0.0007 in² (0.45 mm²) to about 0.01 in² (6.5 mm²) and even more preferably from about 0.001 in² (0.65 mm²) to about 0.002 in² (1.3 mm²).
C. The method according to paragraphs A-B, wherein propellant is added to the antiperspirant composition.
D. The method according to paragraphs A-C, wherein the antiperspirant composition is from about 20% to about 60% solids.
E. The method according to paragraphs A-D, wherein the apparatus further comprises a blade in said mixing chamber disposed opposite the element with an orifice therein.
F. The according to paragraph E, wherein the blade has a Vickers hardness of greater than or equal to about 20 GPa.
G. The method of claim 1, wherein the stream is free of water.
H. The method according to paragraphs A-G, wherein the clay activator is chosen from the group consisting of propylene carbonate, triethyl citrate, methanol, ethanol, acetone, derivatives thereof, or mixtures thereof.
I. The method according to paragraphs A-G, wherein the anhydrous solvent is dimethicone.
J. The method according to paragraph I, wherein the dimethicone is a combination of 5 centistoke dimethicone and 50 centistoke dimethicone.
K. The method according to paragraphs A-J, wherein the hydrophobically modified clay is an organoclay chosen from modified bentonite, modified hectorite, modified montorlinite, or mixtures thereof.
L. The method according to paragraphs A-K, wherein the apparatus's energy density is from about 3.45×10⁶ to about 3.45×10⁷ J/m³.
M. The method according to paragraphs A-L, wherein the pressure at the orifice is greater than 1000 psig.
N. The method according to paragraphs A-M, wherein the pressure at the orifice is from about 1000 psig to about 5000 psig.
O. The method according to paragraphs A-G, wherein the antiperspirant composition comprises a viscosity of from about 2000 centipoise to about 15,000 centipoise at 25° C., preferably from about 2000 centipoise to about 5000 centipoise at 25° C.
P. The method according to paragraphs A-G, wherein late product differentiation material comprises one or more perfumes.

The term “viscosity” means dynamic viscosity (measured in centipoise, cPs, or Pascal-second, Pa·s) or kinematic viscosity (measured in centistokes, cst, or m²/s) of a liquid at approximately 25° C. and ambient conditions. Dynamic viscosity may be measured using a rotational viscometer, such as a Brookfield Dial Reading Viscometer Model 1-2 RVT available from Brookfield Engineering Laboratories (USA) or other substitutable model known in the art. Typical Brookfield spindles which may be used include, without limitation, RV-7 at a spindle speed of 20 rpm, recognizing that the exact spindle may be selected as needed by one skilled in the art. Kinematic viscosity may be determined by dividing dynamic viscosity by the density of the liquid (at 25° C. and ambient conditions), as known in the art.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of making an antiperspirant composition, the method comprising:
a. combining a first group of components comprising an antiperspirant active, a hydrophobically-modified clay, an anhydrous solvent, and a clay activator in a stream;
b. transferring the stream through an apparatus for mixing liquids by producing shear and cavitation, said apparatus comprising:
i. a mixing chamber, said mixing chamber comprising an entrance, at least one inlet, and an outlet; and
ii. an element with an orifice therein, said element being located adjacent the entrance of said mixing chamber, wherein said orifice is configured to produce shear or cavitation in the second stream,
wherein the pressure at the orifice is greater than 500 psig;
wherein the apparatus increases the viscosity of the stream forming the antiperspirant neuter;
c. adding one or more late product differentiation materials to form the antiperspirant composition.

2. The method of claim 1, wherein the size of the orifice is from about 0.0007 in² to about 0.02 in².

3. The method of claim 2, wherein the size of the orifice is from about 0.0007 in² to about 0.01 in².

4. The method of claim 3, wherein the size of the orifice is from about 0.001 in² to about 0.002 in².

5. The method of claim 1, wherein propellant is added to the antiperspirant composition.

6. The method of claim 1, wherein the antiperspirant composition is from about 20% to about 60% solids.

7. The method of claim 1, wherein the apparatus further comprises a blade in said mixing chamber disposed opposite the element with an orifice therein.

8. The method of claim 7, wherein the blade has a Vickers hardness of greater than or equal to about 20 GPa.

9. The method of claim 1, wherein the stream is free of water.

10. The method of claim 1, wherein the clay activator is chosen from the group consisting of propylene carbonate, triethyl citrate, methanol, ethanol, acetone, derivatives thereof, or mixtures thereof.

11. The method of claim 1, wherein the anhydrous solvent is dimethicone.

12. The method of claim 11, wherein the dimethicone is a combination of 5 centistoke dimethicone and 50 centistoke dimethicone.

13. The method of claim 1, wherein the hydrophobically modified clay is an organoclay chosen from modified bentonite, modified hectorite, modified montorlinite, or mixtures thereof.

14. The method of claim 1, wherein the apparatus's energy density is from about 3.45×10⁶ to about 3.45×10⁷ J/m³.

15. The method of claim 1, wherein the pressure at the orifice is greater than 1000 psig.

16. The method of claim 1, wherein the pressure at the orifice is from about 1000 psig to about 5000 psig.

17. The method of claim 1, wherein the antiperspirant composition comprises a viscosity of from about 2000 centipoise to about 15000 centipoise at 25° C.

18. The method of claim 17, wherein the antiperspirant composition comprises a viscosity of from about 2000 centipoise to about 5000 centipoise at 25° C.

19. The method of claim 1, wherein late product differentiation material comprises one or more perfumes.
